(19) United States
(12) Patent Application Publication
(10) Pub. No.: US 2024/0272161 A1
(43) Pub. Date: Aug. 15, 2024

(54) METHOD OF PREDICTING RESPONSE TO IMMUNOTHERAPY

(71) Applicant: The Johns Hopkins University, Baltimore, MD (US)

(72) Inventors: Janis M. Taube, Baltimore, MD (US); Sandor Szalay, Baltimore, MD (US); Andrew M. Pardoll, Baltimore, MD (US); Elizabeth L. Engle, Baltimore, MD (US); Sneha Berry, Baltimore, MD (US); Benjamin Green, Baltimore, MD (US)

(21) Appl. No.: 18/568,444

(22) PCT Filed: Jun. 9, 2022
(86) PCT No.: PCT/US22/32802

§ 371 Date: Dec. 8, 2023

Related U.S. Application Data
(60) Provisional application No. 63/208,829, filed on Jun. 9, 2021.

(51) Int. Cl. G01N 33/574; G01N 33/58; G01N 33/68

(52) U.S. Cl. CPC G01N 33/57484; G01N 33/57423; G01N 33/5743; G01N 33/582; G01N 33/6854; G01N 2333/70517; G01N 2333/70596; G01N 2800/52

(57) ABSTRACT

Provided herein are method of predicting a subject's response to immunotherapy that include (a) staining a biological sample disposed on a substrate: (b) imaging the biological sample, wherein one or more image(s) of a high-power field (HPF) is generated: (c) detecting multiple biomarkers in the biological sample; and (d) analyzing the one or more image(s), thereby predicting the subject's response to immunotherapy.

### CROSS-REFERENCE TO RELATED APPLICATIONS

This application claims priority to U.S. Provisional Patent Application No. 63/208,829, filed on Jun. 9, 2021. The disclosure of this prior application is considered part of the disclosure of this application, and is incorporated in its entirety into this application.

### FEDERALLY SPONSORED RESEARCH OR DEVELOPMENT

This invention was made with government support under grant CA142779 awarded by the National Institutes of Health. The government has certain rights in the invention.

### TECHNICAL FIELD

The present disclosure relates to the field of biotechnology, and more specifically, to assays for tissue-based biomarkers for immunotherapy.

### BACKGROUND

Patients with multiple solid cancer types have shown higher rates of tumor regression and improved survival following treatment with immune checkpoint blocking agents. Unfortunately, for the majority of cancer types, less than half of patients respond to anti-PD-(L)1 agents, and thus it is critical to develop predictive biomarkers that can precisely guide therapy for each patient. PD-L1 immunohistochemistry (IHC) in pre-treatment tumor biopsies is a common tissue-based biomarker approach for predicting response to anti-PD-(L)1, with numerous companion diagnostic indications; however, its expression as a single marker has limited predictive power. Other approaches can also include assessment of microsatellite instability, testing tumor mutational burden, detecting an interferon (IFN)-gamma gene signature, and quantifying multiple proteins by multiplex immunofluorescence (mIF)/IHC. In a recent meta-analysis mIF/IHC demonstrated improved diagnostic performance over other tissue-based approaches when predicting response to anti-PD-(L)1, highlighting the biomarker potential of these emerging technologies.

### SUMMARY

The present disclosure is based on the discovery that predicting a subject's response to immunotherapy as described herein, can be determined by detecting multiple proteins as markers for immune cell type and function simultaneously with a tumor cell marker in a fixed tumor sample using immunofluorescence and/or immunohistochemistry methods. The method in particular analyzes levels of expression of certain of these markers as well as coexpression of markers on individual cells. Without wishing to be bound by any theory, it has been discovered that analysis of biomarkers in the biological sample can be used to predict the subject's response to immunotherapy.

Provided herein are methods of predicting a subject's response to immunotherapy, the method comprising: (a) staining a biological sample disposed on a substrate; (b) imaging the biological sample, wherein one or more image(s) of a high-power field (HPF) is generated; (c) detecting multiple biomarkers in the biological sample; and (d) analyzing the one or more image(s), thereby predicting the subject's response to immunotherapy.

Also provided herein are methods of stratifying a subject and placing the subject in a therapy category, the method comprising: (a) staining a biological sample disposed on a substrate; (b) imaging the biological sample, wherein one or more image(s) of a high-power field (HPF) is generated; (c) detecting multiple biomarkers in the biological sample; and (d) analyzing the one or more image(s), thereby stratifying the subject and placing the subject in a therapy category.

In some embodiments, the multiple biomarkers comprise PD-1, PD-L1, CD8, FoxP3, CD163, a tumor cell marker, or any combination thereof. In some embodiments, the tumor cell marker comprises Sox10, S100, or both.

In some embodiments, the staining comprises an immunofluorescence stain. In some embodiments, the staining comprises an immunohistochemistry stain. In some embodiments, the biological sample is stained with an antibody. In some embodiments, the antibody is a monoclonal antibody. In some embodiments, the antibody is a polyclonal antibody. In some embodiments, the biological sample is stained with one or more antibodies. In some embodiments, the biological sample is stained with six antibodies. In some embodiments, the biological sample is stained with four antibodies. In some embodiments, the biological sample is stained with a second antibody which detects the antibody. In some embodiments, the second antibody is conjugated to a label. In some embodiments, the label is a detectable label. In some embodiments, the label is a fluorophore. In some embodiments, the imaging step (c) comprises performing immunofluorescence microscopy on the biological sample.

In some embodiments, the analyzing step (d) comprises: (i) image acquisition and processing; (ii) cell segmentation and phenotyping; and (iii) image normalization. In some embodiments, the step of image acquisition comprises compiling the one or more images to acquire an image of the whole biological sample within the substrate. In some embodiments, the compiling comprises aligning the one or more images with an overlap.

In some embodiments, the step of cell segmentation and phenotyping comprises identifying a cell type in the biological sample. In some embodiments, the step of phenotyping comprises detecting expression of at least one of the biomarkers in the cell type. In some embodiments, the expression of the at least one biomarker is designated as low, medium, or high. In some embodiments, the cell type comprises a CD163+macrophage, a CD8+ T cell, a Treg cell (CD8negFoxP3+), a tumor cell, a CD8+FoxP3+ cell, or any combinations thereof. In some embodiments, the cell type of CD8+FoxP3+PD-1 low/mid is identified as an indicator that the subject will respond to the immunotherapy. In some embodiments, the cell type of CD163+PD-L1 neg is identified as an indicator that the subject will not respond to the immunotherapy to the same extent as a reference subject that is identified as not having a cell type of CD163+PD-L1 neg.

In some embodiments, the step of cell segmentation and phenotyping further comprises determining a density of the cell type in the biological sample. In some embodiments, the density of the cell type in the biological sample is determined by analyzing a distance between a cell and another cell. In some embodiments, the density of the cell type in the biological sample is determined by analyzing a distance between a cell and a tumor-stromal boundary. In some embodiments, a high density of CD8+FoxP3+ cells is identified as an indicator that the subject will respond to the immunotherapy.

In some embodiments, the step of image normalization comprises calibrating a fluorescence intensity of at least one of the biomarkers in the one or more images against a tissue micro array.

In some embodiments, the analyzing step (c) further comprises identifying the at least one biomarker in the biological sample from a subject having a disease, and wherein the identification of the at least one biomarker is used to predict the subject's response to immunotherapy and/or stratify the subject and place the subject in a therapy category. In some embodiments, the disease is a cancer. In some embodiments, the cancer is a metastatic solid tumor. In some embodiments, the cancer is a melanoma. In some embodiments, the cancer is a non-small cell lung cancer. In some embodiments, the cancer is selected from a bladder cancer, breast cancer, cervical cancer, colon cancer, endometrial cancer, esophageal cancer, fallopian tube cancer, gall bladder cancer, gastrointestinal cancer, head and neck cancer, hematological cancer, Hodgkin lymphoma, laryngeal cancer, liver cancer, lung cancer, lymphoma, melanoma, mesothelioma, ovarian cancer, primary peritoneal cancer, salivary gland cancer, sarcoma, stomach cancer, thyroid cancer, pancreatic cancer, renal cell carcinoma, glioblastoma and prostate cancer.

In some embodiments, the immunotherapy comprises administration of an immune checkpoint inhibitor. In some embodiments, the therapy category comprises radiation therapy, chemotherapy, immunotherapy, hormone therapy, antibody therapy, or any combination thereof.

In some embodiments, the substrate is a slide. In some embodiments, the biological sample comprises a tissue, a tissue section, an organ, an organism, an organoid, or a cell culture sample. In some embodiments, the tissue is a formalin-fixed paraffin-embedded (FFPE) tissue. In some embodiments, the biological sample is fixed prior to step (a). In some embodiments, the biological sample is fixed with formaldehyde. In some embodiments, the biological sample is fixed with methanol.

Also provided herein are methods of improving predictive value of a biomarker, the methods comprising: (a) obtaining a plurality of images of a high-power field (HPF) generated from a biological sample; (b) detecting a biomarker in each of the plurality of images; (c) selecting a sub-plurality of images from the plurality of images of step (a); and (d) analyzing the sub-plurality of images, thereby improving predictive value of the biomarker.

In some embodiments, the method further comprises generating an area under the ROC (receiver operating characteristics) curve value that is greater than an area under the ROC (receiver operating characteristics) curve value generated when analyzing all of the images.

In some embodiments, the biomarker comprises PD-1, PD-L1, CD8, FoxP3, CD163, a tumor cell marker, or any combination thereof. In some embodiments, the tumor cell marker comprises Sox10, S100, or both.

In some embodiments, the sub-plurality of images is 30% of the plurality of images of step (a). In some embodiments, the obtaining step (a) comprises performing immunofluorescence microscopy on the biological sample.

In some embodiments, the analyzing step (d) comprises: (i) image acquisition and processing; (ii) cell segmentation and phenotyping; and (iii) image normalization. In some embodiments, the step of image acquisition comprises compiling the plurality of images of a high-power field (HPF) to acquire an image of the whole biological sample. In some embodiments, the compiling comprises aligning the plurality of images with an overlap.

In some embodiments, the step of cell segmentation and phenotyping comprises identifying a cell type in the biological sample. In some embodiments, the step of phenotyping comprises detecting expression of the biomarker in the cell type. In some embodiments, the expression of the biomarker is designated as low, medium, or high. In some embodiments, the cell type comprises a CD163+macrophage, a CD8+ T cell, a Treg cell (CD8negFoxP3+), a tumor cell, a CD8+FoxP3+ cell, or any combinations thereof. In some embodiments, the cell type of CD8+FoxP3+PD-1 low/mid is identified as an indicator that the subject will respond to the immunotherapy. In some embodiments, the cell type of CD163+PD-L1 neg is identified as an indicator that the subject will not respond to the immunotherapy to the same extent as a reference subject that is identified as not having a cell type of CD163+PD-L1 neg.

In some embodiments, the step of cell segmentation and phenotyping further comprises determining a density of the cell type in the biological sample. In some embodiments, the density of the cell type in the biological sample is determined by analyzing a distance between a cell and another cell. In some embodiments, the density of the cell type in the biological sample is determined by analyzing a distance between a cell and a tumor-stromal boundary. In some embodiments, a high density of CD8+FoxP3+ cells is identified as an indicator that the subject will respond to the immunotherapy.

In some embodiments, the step of image normalization comprises calibrating a fluorescence intensity of the biomarker in the plurality of images against a tissue micro array.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 shows an exemplary schematic of the AstroPath platform for staining optimization and image processing to generate high quality data sets. The optimization of a 6-plex assay for characterizing PD-1 and PD-L1 expression (PD-1, PD-L1, CD163, FoxP3, CD8, Sox10/$100, DAPI) is used to detail the TSA-based AstroPath workflow of multiplex IF with imaging and associated data usage. Solutions to common limitations and sources of error are outlined.

FIGS. 2A-2E show optimization of staining to achieve high sensitivity and specificity using chromogenic IHC. FIG. 2A shows staining index (SI) and bleed-through (BT) propensity that are used to inform TSA fluorophore-marker pairing. FIG. 2B shows sensitivity of IF staining compared to chromogenic IHC, wherein the original signal was decreased in PD-1, PD-L1 and FoxP3 when using the manufacturer's recommended protocol. The sensitivity was increased by replacing the secondary antibody. FIG. 2C shows a graph wherein primary antibody dilutions are then performed to optimize the signal to noise (S/N) ratio, wherein it is indicated that 1:100 is the optimal dilution for CD8 IF staining. FIG. 2D shows the optimal concentration for each TSA fluorophore. Only dilutions with equivalent signal to chromogenic IHC (light grey bars) were considered to ensure sensitivity of the assay. To minimize BT between channels, the lowest acceptable TSA concentration was chosen for the markers (e.g., CD8/540). However, where a fluorophore-marker pair is prone to receive BT, the highest acceptable TSA concentration is chosen to raise the threshold of true positivity (e.g., FoxP3/570). FIG. 2E shows the detection of each marker in multiplex IF compared with its respective monoplex IF, for final validation, confirming equivalence.

FIGS. 3A-3C shows minimization of instrumental errors during field acquisition and the stitching of whole slide using lessons from astronomy. FIG. 3A shows an image of the entire tissue of interest captured using HPFs with 20% overlap as shown in the low and high power images (average 1300 fields acquired per case). FIG. 3B shows an image wherein each HPF was found to have instrumental imaging errors, including lens distortion and variations in field illumination. FIG. 3C shows pixels in overlapping image regions that were compared to determine the field alignment error. In order to improve alignment, a spring-based model was used to minimize pixel shift. The misalignment error was reduced from +/−3 pixels in the x-direction and from +/−5 pixels in the y-direction, to less than −/−1 pixel for both (ranges were reported for the 95th-5th percentile). The illumination variation was also reduced, from a 11.2% variance to 1.2% variance.

FIGS. 4A-4B show immune cell populations and marker expression in situ vary by location. FIG. 4A shows a representative mIF image showing a hot-spot at the edge of the tumor with T-cells showing PD-1low expression adjacent to cells with PD-L1high expression. Within the tumor parenchyma, cells that are PD-1high and PD-1mid were observed, adjacent to PD-L110″ expression consistent with a more exhausted T-cell phenotype. Histograms including all cases in the cohort show cell densities of CD8+ cells displaying PD-1 as a function of distance to tumor boundary. PD-1 expression intensity increased as T-cells were exposed to tumor antigen. FIG. 4B shows a representative image of a metastatic melanoma deposit showing localization of CD8+FoxP3+ cells in areas of dense CD8+PD-1neg and CD8+PD-1+ cell infiltrates, adjacent to tumor cells demonstrating adaptive (IFN-gamma-driven) PD-L1 expression by tumor. Histograms including all cases in the cohort show that CD8+FoxP3+ cells are most likely to be localized near CD8+PD-1^{neg} cells. Other cell types in the same relative location to the tumor-stromal boundary include CD8+PD-1+ cells and PD-L1+tumor cells.

FIGS. 5A-5B show AUC heat maps for response to therapy as a function of various immune cell types expressing PD-1/L1 and the intensity of PD-1/L1 expression using two different slide sampling strategies. FIG. 5A shows PD-1/PD-L1 mIF assay combined with hot-spot HPF selection showing that the densities of CD8+FoxP3+PD-1^{low/mid}, Tumor PD-L1^{neg} and CD163+PD-L1^{neg} cells have the highest value of individual features for predicting response and non-response to anti-PD-1. Approximately 86% of CD8-FoxP3-PD-1^{pos} cells in melanoma represent conventional CD4T-cells. FIG. 5B shows a similar characterization using representative field sampling, wherein similar key features associated with response to therapy were highlighted. However, the resultant AUCs, particularly for the CD8+ cell subsets, were not as high using this approach. This finding highlights the fact that slide sampling is another component of assay performance. It is also an element that can be optimized and standardized. TumorPD-L1^{neg} and CD163+PD-L1^{neg} are negatively associated features, all others are positively associated features.

FIGS. 6A-6D show multifactorial analysis of 6-plex mIF assay with a focus on PD-1 and PD-L1 intensities for predicting objective response and long-term survival. FIG. 6A is a table showing the ten features associated with response to therapy by univariate analysis at 30% hot spot HPFs. Features are listed in decreasing order of predictive value. FIG. 6B show combinatorial ROC curves and the corresponding AUC values were assessed for these 10 features in the Discovery cohort, as well as a second, independent cohort. FIG. 6C show TMEs from patients, wherein poor prognosis is characterized by high densities of tumor cells and CD163+ cells that lack PD-L1 expression, irrespective of whether other immune cells are present (left panel). Those with intermediate prognosis have TMEs with low level (middle panel) immune infiltrates and are not CD163+PD-L1^{neg} myeloid-rich. The patients with the best prognosis have TMEs that are highly inflamed, e.g., CD8+ and CD8+FoxP3+ T-cells expressing various levels of PD-1 and PD-L1 (right panel). PD-L1 expression is also evident on CD163+ cells. FIG. 6D shows graphs wherein distinct TMEs defined by specific cell types displaying various levels of PD-1 and PD-L1 stratified patients into those with poor, intermediate, and good overall survival (OS) and progression free survival (PFS) in a discovery cohort, Kaplan-Meier analysis. Similar stratification of patient outcomes was achieved using an independent, validation cohort from a different institution (OS, p=0.036; PFS, p=0.024, log-rank test).

FIGS. 7A-7C show an exemplary schematic of Tyramide signal amplification (TSA) technology that can be used to amplify signal and visualize multiple markers on a single slide. FIG. 7A is an exemplary schematic showing that TSA detection allows for greater amplification (˜1000 fold) of signal when compared to staining using a fluorophore tagged secondary antibody. This ability can be attributed to the deposition of multiple TSA fluorophore molecules by an enzyme catalyzed reaction. FIG. 7B shows a multiplex staining process that can be broken down in to three phases: slide preparation, sequential staining and final processing. FIG. 7C shows an exemplary schematic showing, in the sequential staining phase, microwave treatment (MWT) strips off antibodies from prior staining rounds while retaining the deposited TSA fluorophore due its stronger binding to the tissue. The staining process can be repeated for multiple markers without any cross-reactivity.

FIG. 8 shows multispectral image acquisition using the Vectra 3.0 system that allows for the simultaneous visualization of six channels of interest plus DAPI. A mercury-halogen lamp emits light that is received by five excitation cubes whose wavelengths span the visible spectrum. Light is next received by the liquid crystal tunable filter which allows specific wavelengths to pass through, each one forming an individual monochromatic image plane. The resultant images are then unmixed using a library of pure spectra for each fluorophore. The individual images for each fluorophore are then pseudo-colored and overlayed to form a composite image. Unmixed images are then further processed using inFormTM software.

FIGS. 9A-9B show characterization of TSA fluorophores for stain index (SI) and bleed-through (BT). FIG. 9A shows that the SI is a signal to background metric useful for quantifying the brightness of immunofluorescent reagents. Fluorophores 540 and 620 had the lowest and the highest SIs respectively. TSA fluorophores with lower SIs were paired with more abundant markers e.g. CD8 (an abundant, strong antigen) is paired with Opal 540 (a fluorophore with a low SI). FIG. 9B shows that BT can be the detection of false positive signal in a channel due to spillover from a different channel. The propensity for BT of each fluorophore, when used at a dilution of 1:50, was characterized. Top left: The logarithm of the normalized intensity of fluorescence for each possible TSA fluorophore-TSA fluorophore pair was plotted. A parameterized hyperbolic sine curve was fitted as shown on the graph. Top right: Table shows the propensity of BT from each channel to another, i.e., A*a in the parameterized hyperbolic sine function. The most significant BT between fluorophores ranked from high to low are: 540 to 570, 650 to 620, 520 to 570, 540 to 620 and 540 to 520. Bottom left: Examples of low and high BT. Bottom right: Representative 540 to 570 BT is shown in the photomicrographs where membranous signal from CD8 cells is seen in the 570 (FoxP3) channel (real FoxP3 staining is nuclear). The propensity of BT is further reduced by diluting the TSA fluorophores during subsequent steps of panel optimization.

FIGS. 10A-10B show that primary antibody optimization is required to maximize IF staining specificity using chromogenic IHC as the gold standard. After selection of the appropriate HRP-conjugated polymer, primary antibody dilutions were performed to optimize the signal to noise (S/N) ratio, i.e., the specificity. FIG. 10A show a representative figure for CD8 monoplex IF staining indicating that 1:100 is the dilution with the optimal S/N ratio. Concordance was seen between three different approaches for signal quantification. FIG. 10B shows that when the appropriate HRP-conjugated polymer is paired with the optimal primary antibody concentration, monoplex IF yields equivalent signal to chromogenic IHC.

FIGS. 11A-11B show a comparison of monoplex IF and multiplex IF staining. FIG. 11A show bar graphs showing that when optimized as detailed herein, multiplex IF yields an equivalent percentage of positive cells to monoplex IF. FIG. 11B shows that the usable dynamic range of the epitope was reduced by 13% on average in multiplex IF format. Each dot represents the difference between the 95th percentile and 5th percentile of the mean normalized fluorescence intensity of positive cells for a single HPF.

FIGS. 12A-12C show that the optimum overlap of neighboring tiles is x=20% of the tile width and height. FIG. 12A show that overlapping image tiles (examples shown in red) are used to create a seamless coverage of the whole area, built from the central rectangles of each image (blue lines, with peach shading showing one central rectangle). These central rectangles form the statistical sample for analysis, and fully cover the tissue. The overlaps (areas shaded in darker blue), are observed multiple times and are used for intrinsic error estimates. FIG. 12B show that too much overlap is “costly” in terms of data resources and time, while too little overlap fails to provide enough information to correct for imaging deficiencies. The information content (inverse variance) in estimating the corrections is proportional to the areas T and O, respectively. In the equation, the useful area is T, representing the tissue area on the slide, and O is the area of the overlaps. FIG. 12C shows that the optimum solution is x=0.2, i.e. 20%, corresponding to the case when the area that is imaged multiple times (O) is equal to the area of the tissue itself (T).

FIGS. 13A-13C show that image processing of individual fields included flatfield corrections for systematic illumination variation. FIG. 13A shows an average of 11,508 images were stacked to define the average illumination variation by image layer across a single HPF. Shown is the uncorrected, smoothed mean image for layer 13 (FITC broad band filter, PD-L1). FIG. 13B shows that a flatfield model was developed and applied, and the resultant smoothed, corrected mean image is shown. FIG. 13C shows relative pixel intensities between uncorrected and corrected images showing a consistent 9-fold reduction of illumination variation (11.2% to 1.2% for the 5th-95th percentile and 3.6% to 0.4% standard deviation on average). Pixel intensities relative to mean layer intensities are shown here across all image layers for one representative sample.

FIGS. 14A-14C show image tiles generated using the 20% overlap approach are stitched to an absolute Cartesian coordinate system, creating a whole slide image that is accurate to a fraction of a pixel without loss of information. FIG. 14A shows that simple abutting of image tiles potentially contributes to a loss of reliable information for approximately 3-6% of cells. FIG. 14B shows a schematic visualization of how jumps in mechanical stage movement and inaccuracies in the underlying stitching algorithms accumulate in the x and y direction across a slide. The relative displacements in the x and y direction required to seamlessly stitch image tiles are denoted as dx and dy. It was found that on average the cumulative shift across a whole slide contributes 20 μm error in both the x and y direction. FIG. 14C shows the contours from uncorrected stitching, overlaid on images generated using the AstroPath approach. Uncorrected whole slide stitching contributed up to an ˜80 pixel shift which equates to 40 μm or the diameter of 4 lymphocytes. Correcting such errors will be especially important when multiple microscopes, software analysis suites, and/or scans from different systems are used. Such directional, cumulative shifts could also contribute to inaccuracies in slide registration when Z-stacking images (i.e. overlaying a second slide image on top of the first image from the same specimen).

FIGS. 15A-15D show that single-marker phenotyping approach minimizes error in dataset due to over-segmentation of large cells. FIG. 15A shows representative images displaying improved cell segmentation using a single-marker approach (red lines=cell boundaries, *=over-split tumor nuclei). FIG. 15B shows a representative image of merged phenotype output following single-marker phenotyping (top left), and a corresponding output of each individual single-marker phenotype algorithm before merging (bottom and right). FIG. 15C shows the number of positive cells quantified by the single-marker approach reflecting the ‘gold standard’, while the multi-marker approach overestimates the tumor and CD163+ cells. The ‘gold standard’ is defined as segmentation/phenotyping performed for each lineage marker on monoplex IF (i. e. individual stain). FIG. 15D is a table wherein this systematic error was further characterized by testing the number of cells counted in CD8 hotspots from 46 specimens by the single-marker and multi-marker approaches. Percent differences between cell counts show the multi-marker approach leads to a 30% over counting of tumor and CD163 cells, compared to the single-marker approach.

FIGS. 16A-16E show representative output from bespoke algorithms that facilitate visual inspection of segmentation and phenotyping performance. FIG. 16A shows a custom display that shows ˜1250 cells per view. A colored dot is placed on each cell in the mIF image indicating the lineage. Additionally, a dash is placed over the cell if PD-L1 (green dash) and/or PD-1 (cyan dash) is expressed. 20 views per specimen were visually inspected, except for the rare cases with less tissue availability. FIG. 16B shows the inspection of up to 25 randomly selected positive and negative cells/stamps for each marker across all HPFs in a given specimen. The results of the segmentation algorithm are shown in red, and each cell that is positive for a given marker is labeled with a white “+”. A minimum of 2000 cells displaying each marker was visually inspected per specimen using these stamps. FIG. 16C shows additional representative QA/QC stamps without the overlying cell segmentation map. The “+” shows each cell that was called positive by the algorithm. FIG. 16D shows that the QA/QC stamp viewer can also be used to visually inspect co-expression profiles of interest. Representative images of CD8+FoxP3+ cells are shown (FoxP3 in red; CD8 in yellow). An average of 200 CD8+FoxP3+ cells per specimen were visually inspected. FIG. 16E shows three examples of CD8+ cells (yellow) that are PD-L1+(green). The three images were obtained from three different patient specimens to show generalizability. The top row shows the CD8 channel only; the middle row shows the PD-L1+channel only; and the bottom row shows the CD8 and PD-L1 channels together. Specifically, the left panel in the bottom row shows a cell that is CD8+PD-L1-cell (single yellow asterisk), a cell that is CD8-PD-L1+ (single green asterisk), and a cell that is CD8-PD-L1+(one yellow and one green asterisk). The DAPI is also displayed in blue.

FIGS. 17A-17B show that accurate comparison of specimens stained at different times requires the correction of batch-to-batch variation. FIG. 17A shows graphs wherein batch-to-batch variation was evident for PD-1 and PD-L1 expression intensities. It was corrected through normalization to a tissue microarray slide containing tonsil and spleen (n=3 each), which was run with each batch. FIG. 17B shows a bar graph wherein the percent coefficient of variation across the 9 batches was 17% for PD-1 and 22% for PD-L1, and was reduced by ˜50% for both markers once normalized.

FIGS. 18A-18B show PD-(L)1^{low}, PD-(L)1^{mid} and PD-(L)1^{high} intensity levels. FIG. 18A is a histogram showing PD-1 (left) and PD-L1 (right) intensity cut-offs that were defined by pooling all PD-1^{pos} or PD-L1^{pos} cells and dividing the population into tertiles. FIG. 18B shows photomicrographs (brightfield on top and IF on bottom) showing the location of PD-1+ populations vary by specific regions in tonsil tissue (left). PD-1^{high} cells are predominantly located in germinal center T-cells in the light zone, while PD-1^{low} and PD-1^{mid} cells are found in the interfollicular zone. Photomicrographs (brightfield on top and IF on bottom) showing the location of PD-L1+ populations also vary by microanatomic location within tonsil (right). The tonsillar crypts show PD-L1^{high} cells. PD-L1^{mid} and PD-L1^{low} cells are observed in the germinal centers, and scattered PD-L1^{low} perifollicular cells may also be seen. For assay optimization of PD-1 and PD-L1 signal in the mIF assay, anatomic regions of low and mid expression were preferentially selected.

FIGS. 19A-19B show densities of specific cell populations in responders vs. non responders across the entire TME. The mean tumor area analyzed among the 53 patients was 61 mm² (range 5-308 mm²). FIG. 19A shows that there was no significant difference in densities of PD-L1 positive cells between responders and non-responders to anti-PD-1 when scoring for % tumor cell expression using the commercially available chromogenic 22C3 IHC assay and interpreted by a pathologist using light microscopy. Representative photomicrograph of PD-L1 IHC shown on right. FIG. 19B shows that total and tumor cell PD-L1+ cell densities across the entire TME (whole slide analysis using 6-plex mIF assay on the AstroPath platform) were associated with response while no significant associations were seen for CD163+PD-L1+ cell densities. Median +/−95% CI, one-tailed Mann-Whitney. Representative photomicrographs shown in right column with PD-L1 on any cell type by mIF assay (top row), PD-L1 and tumor (middle row), and PD-L1 and CD163 (bottom row).

FIG. 20A shows PD-1 expression proportion within the melanoma TME by cell type. 94 archival melanoma specimens in TMA format were stained using a mIF assay for PD-1, CD8, CD4, CD20, FoxP3, and tumor (Sox10/S100). CD8+ cells contributed the majority of PD-1 to the melanoma TME. Of the non-CD8+ cells contributing PD-1, 86% labeled as CD4+(65% of conventional CD4+ cells and 21% of CD4+FoxP3+ cells).

FIG. 20B shows a photomicrograph of a representative CD20-PD-1+ cell.

FIGS. 21A-21B show that analysis of the whole TME (100% sampling) was not as effective at stratifying patients as 30% sampling, when similar strategies were applied. At 100% TME sampling, the features with AUCs having p-values <0.05 after correction for multiple tests were identified (positive features: CD8+PDL1^{low}, CD8+FoxP3+, CD8+FoxP3+PD-1^{low} CD8+FoxP3+PD-1^{mid}, tumor PD-L1^{low}, and negative features: CD163+PD-L1^{neg} and CD163+ cells) (Table 11). These features were used to generate combinatorial ROC curves and Kaplan-Meier curves for the Discovery cohort (FIG. 21A), as well as a second, independent Validation cohort (FIG. 21B). Similar general trends were observed to the 30% slide sampling (FIG. 6), though the stratification was not as efficacious. This finding highlights the fact that slide sampling is another component of assay development that can be optimized and standardized.

FIGS. 22A-22B show TMEs defined by specific cell types and association with long-term survival by Kaplan-Meier analysis for smaller specimens. The minimum tumor area for inclusion in the study was 5 mm². Patients where <20 mm² (FIG. 22A) and >20 mm² (FIG. 22B) tumor area was present on the slide are separated into good, intermediate and poor prognosis using scoring rules defined for FIG. 12B. 20 mm² in surface area was chosen because it represents the size of 3 core biopsies (each 1 mm×15 mm in size) with ˜50% tumor in each core.

FIGS. 23A-23B show results from reduction of mIF assay from 6-plex to 4-plex for predicting objective response and stratifying overall survival. In the index 6-plex assay (FIG. 6), CD8+subsets were used to predict patients with good vs. intermediate long-term outcomes. Here, we tested whether total CD8+ cell densities alone could be used for this distinction, potentially reducing the number of requisite markers from 6 to 4 (CD8, CD163, PD-L1, Sox 10/S100). These four features were used to generate combinatorial ROC curves and Kaplan-Meier curves for the Discovery cohort (FIG. 23A) and Validation cohort (FIG. 23B). It was found that the highest tertile of total CD8+densities, when combined with the highest densities of features negatively associated with response (CD163+PD-L1^{neg}, Tumor PD-L1^{neg} or tumor cells), could be combined to stratify survival. This approach is not as efficacious for predicting outcomes, especially with regard to predicting objective response. However, it has the advantage of allowing for the inclusion of additional markers, e.g. markers that could either help resolve the patients in the intermediate prognosis group or to help identify factors within the TME from patients in the intermediate and poor prognostic categories to help inform new, rational treatment strategies.

FIG. 24 shows CD8+FoxP3+PD-1+ cells strongly associate with objective response for patients with advanced non-small cell lung cancer treated with anti-PD-1-based therapy. Pre-treatment lung cancer specimens from n=20 patients with advanced disease were stained with a 6-plex mIF assay, and the entire specimen was imaged using a tiled mosaic of high power fields (HPFs).

FIGS. 25A-25B show the AstroPath imaging approach was applied to pre-treatment specimens from patients with non-small cell lung cancer receiving anti-PD-1 in the neoadjvuant setting for advanced disease. FIG. 25A shows the median pre-treatment biopsy size in this cohort was 3 mm² (average 15 mm²). FIG. 25B show a second analysis was performed to determine pre-treatment features that predicted the degree of pathologic response to therapy. Left: The heat map shows some potential cell populations identified by this method and their association with pathologic response values of 10% residual viable tumor (rvt10, which is an endpoint of numerous Phase II/III clinical trials) and 50% residual viable tumor (rvt50). Right: The features identified using this approach can then be used to predict survival outcomes for these patients (Kaplan-Meier analysis).

### DETAILED DESCRIPTION

The present disclosure is based on the discovery that analysis of multiple cell types and their spatial interactions, as well as expression levels and cellular profiles of biomarkers (e.g., immunoregulatory molecules) can be used to predict a subject's response to immunotherapy. In some embodiments, detecting one or more biomarkers (e.g., PD-1, PD-L1, CD8, FoxP3, CD163, and a tumor cell marker) in a biological sample using immunofluorescence and/or immunohistochemistry methods can be used to predict response to checkpoint inhibitor therapies (e.g., checkpoint blockade with anti-PD-1-based therapy) and/or stratify long-term survival after the immunotherapy. While immunotherapies (e.g., immune checkpoint inhibitor (ICI) therapies) have transformed cancer care by improving overall survival (OS), much effort is being dedicated to the development of predictive biomarkers, in order to direct specific treatments to patients with the best chance of benefit while seeking alternatives for those patients who are highly unlikely to respond.

In some embodiments, provided herein are methods of predicting a subject's response to immunotherapy that include (a) staining a biological sample disposed on a substrate; (b) imaging the biological sample, wherein an image of a high-power field (HPF) is generated; (c) detecting one or more biomarkers in the biological sample; and (d) analyzing the HPF image, thereby predicting the subject's response to immunotherapy.

Various non-limiting aspects of these methods are described herein, and can be used in any combination without limitation. Additional aspects of various components of the methods described herein are known in the art.

As used herein, the term “administration” typically refers to the administration of a composition to a subject or system to achieve delivery of an agent that is, or is included in, the composition. Those of ordinary skill in the art will be aware of a variety of routes that may, in appropriate circumstances, be utilized for administration to a subject, for example a human. For example, in some embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e.g., intrahepatic), mucosal, nasal, oral, rectal, subcutaneous, sublingual, topical, tracheal (e.g., by intratracheal instillation), vaginal, vitreal, etc. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

As used herein, the term “antibody” refers to an agent that specifically binds to a particular antigen. In some embodiments, the term encompasses any polypeptide or polypeptide complex that includes immunoglobulin structural elements sufficient to confer specific binding. Exemplary antibody agents include, but are not limited to monoclonal antibodies, polyclonal antibodies, and fragments thereof. In some embodiments, an antibody agent may include one or more sequence elements are humanized, primatized, chimeric, etc. as is known in the art. In many embodiments, the term “antibody” is used to refer to one or more of the art-known or developed constructs or formats for utilizing antibody structural and functional features in alternative presentation. For example, in some embodiments, an antibody utilized in accordance with materials and methods provided herein is in a format selected from, but not limited to, intact IgA, IgG, IgE or IgM antibodies; bi- or multi-specific antibodies (e.g., Zybodies®, etc.); antibody fragments such as Fab fragments, Fab′ fragments, F(ab′)2 fragments, Fd′ fragments, Fd fragments, and isolated CDRs or sets thereof; single chain Fvs (scFvs); polypeptide-Fc fusions; single domain antibodies (e.g., shark single domain antibodies such as IgNAR or fragments thereof); cameloid antibodies; masked antibodies (e.g., Probodies®); Small Modular Immunopharmaceuticals (“SMIPs™”); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies; Adnectins®; Affilins®; Trans-Bodies®; Affibodies®; TrimerX®; MicroProteins; Fynomers®, Centyrins®; and KALBITOR®s. In some embodiments, an antibody is or comprises a polypeptide whose amino acid sequence includes structural elements recognized by those skilled in the art as an immunoglobulin variable domain. In some embodiments, an antibody is a polypeptide protein having a binding domain which is homologous or largely homologous to an immunoglobulin-binding domain. In some embodiments, an antibody is or comprises at least a portion of a chimeric antigen receptor (CAR). In some embodiments, an antibody is or comprises a T cell receptor (TCR).

As used herein, the term “biological sample” refers to a sample obtained from a subject for analysis using any of a variety of techniques including, but not limited to, biopsy, surgery, and laser capture microscopy (LCM), and generally includes cells and/or other biological material from the subject. A biological sample can be obtained from a eukaryote, such as a patient derived organoid (PDO) or patient derived xenograft (PDX). The biological sample can include organoids, a miniaturized and simplified version of an organ produced in vitro in three dimensions that shows realistic micro-anatomy. Subjects from which biological samples can be obtained can be healthy or asymptomatic individuals, individuals that have or are suspected of having a disease (e.g., cancer) or a pre-disposition to a disease, and/or individuals that are in need of therapy or suspected of needing therapy.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells.

Biological samples can also include immune cells. Sequence analysis of the immune repertoire of such cells, including genomic, proteomic, and cell surface features, can provide a wealth of information to facilitate an understanding the status and function of the immune system. Examples of immune cells in a biological sample include, but are not limited to, B cells (e.g., plasma cells), T cells (e.g., cytotoxic T cells, natural killer T cells, regulatory T cells, and T helper cells), natural killer cells, cytokine induced killer (CIK) cells, myeloid cells, such as granulocytes (basophil granulocytes, eosinophil granulocytes, neutrophil granulocytes/hypersegmented neutrophils), monocytes/macrophages, mast cells, thrombocytes/megakaryocytes, and dendritic cells.

The biological sample can include any number of macromolecules, for example, cellular macromolecules and organelles (e.g., mitochondria and nuclei). The biological sample can be a nucleic acid sample and/or protein sample. The biological sample can be a carbohydrate sample or a lipid sample. The biological sample can be obtained as a tissue sample, such as a tissue section, biopsy, a core biopsy, needle aspirate, or fine needle aspirate. The sample can be a fluid sample, such as a blood sample, urine sample, or saliva sample. The sample can be a skin sample, a colon sample, a cheek swab, a histology sample, a histopathology sample, a plasma or serum sample, a tumor sample, a lymph node sample, living cells, cultured cells, a clinical sample such as, for example, whole blood or blood-derived products, blood cells, or cultured tissues or cells, including cell suspensions.

As used herein, the terms “cancer”, “malignancy”, “neoplasm”, “tumor”, and “carcinoma” refer to cells that exhibit relatively abnormal, uncontrolled, and/or autonomous growth, so that they exhibit an aberrant growth phenotype characterized by a significant loss of control of cell proliferation. In some embodiments, a tumor may be or comprise cells that are precancerous (e.g., benign), malignant, pre-metastatic, metastatic, and/or non-metastatic. The present disclosure specifically identifies certain cancers to which its teachings may be particularly relevant. In some embodiments, a relevant cancer may be characterized by a solid tumor. In some embodiments, a relevant cancer may be characterized by a metastatic solid tumor. In some embodiments, a relevant cancer may be characterized by a hematologic tumor.

In general, examples of different types of cancers known in the art include, for example, a bladder cancer, breast cancer, cervical cancer, colon cancer, endometrial cancer, esophageal cancer, fallopian tube cancer, gall bladder cancer, gastrointestinal cancer, head and neck cancer, hematological cancer, Hodgkin lymphoma, laryngeal cancer, liver cancer, lung cancer, lymphoma, melanoma, mesothelioma, ovarian cancer, primary peritoneal cancer, salivary gland cancer, sarcoma, stomach cancer, thyroid cancer, pancreatic cancer, renal cell carcinoma, glioblastoma and prostate cancer. In some embodiments, hematopoietic cancers can include leukemias, lymphomas (Hodgkin's and non-Hodgkin's), myelomas and myeloproliferative disorders; sarcomas, melanomas, adenomas, carcinomas of solid tissue, squamous cell carcinomas of the mouth, throat, larynx, and lung, liver cancer, genitourinary cancers such as prostate, cervical, bladder, uterine, and endometrial cancer and renal cell carcinomas, bone cancer, pancreatic cancer, skin cancer, cutaneous or intraocular melanoma, cancer of the endocrine system, cancer of the thyroid gland, cancer of the parathyroid gland, head and neck cancers, breast cancer, gastro-intestinal cancers and nervous system cancers, benign lesions such as papillomas, precancerous pathology such as myelodysplastic syndromes, acquired aplastic anemia, Fanconi anemia, paroxysmal nocturnal hemoglobinuria (PNH) and 5q-syndrome and the like.

As used herein, the term “therapeutic agent” and “chemotherapeutic agent” can refer to one or more pro-apoptotic, cytostatic and/or cytotoxic agents, for example specifically including agents utilized and/or recommended for use in treating one or more diseases, disorders or conditions associated with undesirable cell proliferation. In many embodiments, chemotherapeutic agents are useful in the treatment of cancer. In some embodiments, a chemotherapeutic agent may be or comprise one or more alkylating agents, one or more anthracyclines, one or more cytoskeletal disruptors (e.g. microtubule targeting agents such as taxanes, maytansine and analogs thereof, of), one or more epothilones, one or more histone deacetylase inhibitors HDACs), one or more topoisomerase inhibitors (e.g., inhibitors of topoisomerase I and/or topoisomerase II), one or more kinase inhibitors, one or more nucleotide analogs or nucleotide precursor analogs, one or more peptide antibiotics, one or more platinum-based agents, one or more retinoids, one or more vinca alkaloids, and/or one or more analogs of one or more of the following (i.e., that share a relevant anti-proliferative activity). In some embodiments, a chemotherapeutic agent may be utilized in the context of an antibody-drug conjugate.

As used herein, the term “stratify” refers to assigning a treatment regimen. In some embodiments, a subject can be stratified and placed in a therapy category, wherein a treatment regimen in the therapy category is assigned to the subject. In some embodiments, stratification of a subject can be used in prospective or retrospective clinical studies. In some embodiments, stratification of a subject can be used to assign a prognosis or a prediction regarding survival or chemotherapy or radiotherapy sensitivity. In some embodiments, stratification typically assigns a subject to a group based on a shared mutation pattern or other observed characteristic or set of characteristics. In some embodiments, the treatment regimen can be an anti-cancer treatment. In some embodiments, the treatment regimen can be in a treatment category, wherein the treatment category comprises anti-cancer treatments. In some embodiments, the treatment category can include radiation therapy, chemotherapy, immunotherapy, hormone therapy, antibody therapy, or any combination thereof.

As used herein, the term “subject” refers an organism, typically a mammal (e.g., a human, in some embodiments including prenatal human forms). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

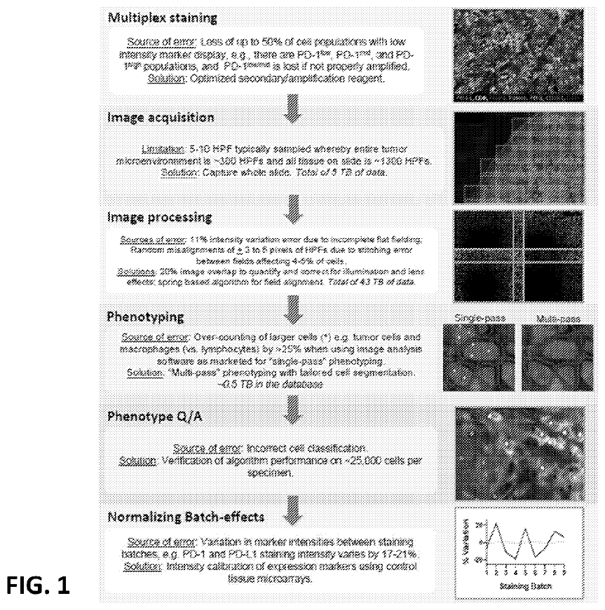

As used herein, the term “treatment outcome” refers to an evaluation undertaken to assess the results or consequences of management and procedures used in combating disease in order to determine the efficacy, effectiveness, safety, and practicability of treatments given to a subject. In some embodiments, the determination of treatment outcome can include whether a subject will respond to the specific treatment administered to the subject. In some embodiments, determination of treatment outcome can be used to stratify patients with a disease into groups with differential treatment outcome (e.g., overall survival rate, disease control rate). In some embodiments, determination of treatment outcome can include analyzing overall survival rate, disease control rate, changes in psychological condition, or changes in physical condition (e.g., tissue damage, pain level). In some embodiments, a subject that exhibits a given cell type (e.g., a CD8+ T cell, a CD8+FoxP3+ cell) is predicted to have an improved outcome as compared to a reference subject that is identified as not having the cell type (FIG. 1).

### Platform for Predicting Response to Immunotherapy

In some embodiments, provided herein are methods of predicting a subject's response to immunotherapy, the method including: (a) staining a biological sample disposed on a substrate;

(b) imaging the biological sample, wherein an image of a high-power field (HPF) is generated; (c) detecting one or more biomarkers in the biological sample; and (d) analyzing the HPF image, thereby predicting the subject's response to immunotherapy.

### Immunotherapy

As used herein, “immunotherapy” refers to a treatment of disease (e.g., cancer) by activating or suppressing the immune system. For example, cancer immunotherapy uses the immune system and its components to mount an anti-tumor response through immune activation. In some embodiments, an immunotherapy can include an immune checkpoint inhibitor, an oncolytic virus therapy, a cell-based therapy, a CAR-T cell therapy, or a cancer vaccine. In some embodiments, an immunotherapy can include immune checkpoint blockade, wherein an immune checkpoint inhibitor is administered. In some embodiments, the immunotherapy includes administration of an immune checkpoint inhibitor. In some embodiments, the immune checkpoint inhibitor is a PD-1 inhibitor. Examples of a PD-1 inhibitor can include, but are not limited to, pembrolizumab, nivolumab, cemiplimab, JTX-4014, spartalizumab, camrelizumab, sintilimab, tislelizumab, toripalimab, and dostarlimab. In some embodiments, the immune checkpoint inhibitor is a PD-L1 inhibitor. Examples of a PD-L1 inhibitor can include, but are not limited to, atezolizumab, avelumab, durvalumab, KN035, CK-301, AUNP12, CA-170, and BMS-986189. In some embodiments, the immune checkpoint inhibitor is a CTLA-4 inhibitor (e.g., ipilimumab, tremelimumab). In some embodiments, the immune checkpoint inhibitor is a CTLA-4 inhibitor used in combination with a PD-1 inhibitor or a PD-L1 inhibitor. In some embodiments, the immune checkpoint inhibitor can be any checkpoint inhibitor, e.g., as described in Mazzarella et al., Eur J Cancer (2019) 117:14-31, hereby incorporated by reference.

### Biomarkers

In some embodiments, detecting one or more biomarkers in the biological sample can be used to predict a subject's response to immunotherapy. In some embodiments, detecting one or more biomarkers in the biological sample can be used to monitor a subject's response to immunotherapy. As used herein, the term “biomarker” refers to a measurable indicator of the severity or presence of a disease (e.g., cancer) state. In some embodiments, a biomarker can be used to help diagnose conditions (e.g., identify early stage cancers). In some embodiments, a biomarker can be used to determine a subject's overall survival rate without treatment or therapy. In some embodiments, a biomarker can predict a subject's response to a treatment (e.g., immunotherapy). In some embodiments, one or more biomarkers can be detected in the biological sample. In some embodiments, the one or more biomarkers can include PD-1, PD-L1, CD8, FoxP3, CD163, a tumor cell marker, or any combination thereof. In some embodiments, a biomarker can include a tumor cell marker. In some embodiments, the tumor cell marker can include AFP, BRAF V600E, S100, Sox10, cytokeratins, Melan-A, HMB45, vimentin, desmin, myogenin, smooth muscle actin, GFAP, synaptophysin, chromogranin, CD45/LCA, or any combination thereof. In some embodiments, the tumor cell marker can be a combination of Sox 10 and S100.

### Multiplex Staining

In some embodiments, the method described herein includes staining a biological sample disposed on a substrate. To facilitate visualization, the biological sample can be stained using a wide variety of stains and staining techniques. In some embodiments, a biological sample can be stained using any number of biological stains, including but not limited to, acridine orange, Bismarck brown, carmine, coomassie blue, cresyl violet, DAPI, eosin, ethidium bromide, acid fuchsine, hematoxylin, Hoechst stains, iodine, methyl green, methylene blue, neutral red, Nile blue, Nile red, osmium tetroxide, propidium iodide, rhodamine, or safranin.

The biological sample can be stained using known staining techniques, including Can-Grunwald, Giemsa, hematoxylin and eosin (H&E), Jenner's, Leishman, Masson's trichrome, Papanicolaou, Romanowsky, silver, Sudan, Wright's, and/or Periodic Acid Schiff (PAS) staining techniques. In some embodiments, the biological sample can be stained by using tyramide signal amplification (TSA) technology. In some embodiments, the biological sample can be stained by using a pan-membrane stain. In some embodiments, the biological sample can be stained by using a plasma membrane stain.

In some embodiments, the staining comprises an immunofluorescence (IF) stain. In some embodiments, the staining comprises an immunohistochemistry (IHC) stain. In some embodiments, the biological sample can be stained using a detectable label (e.g., radioisotopes, fluorophores, chemiluminescent compounds, bioluminescent compounds, and dyes). In some embodiments, a biological sample is stained using only one type of stain or one technique. In some embodiments, staining includes biological staining techniques such as H&E staining. In some embodiments, staining includes using fluorescently-conjugated antibodies. In some embodiments, a biological sample is stained using two or more different types of stains, or two or more different staining techniques. For example, a biological sample can be prepared by staining and imaging using one technique (e.g., H&E staining and brightfield imaging), followed by staining and imaging using another technique (e.g., IHC/IF staining and fluorescence microscopy) for the same biological sample.

Methods for multiplexed staining are described, for example, in Bolognesi et al., J. Histochem. Cytochem. 2017; 65(8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

In some embodiments, the biological sample is stained with an antibody. In some embodiments, the antibody is a monoclonal antibody. In some embodiments, the antibody is a polyclonal antibody. In some embodiments, the biological sample is stained with one or more antibodies (e.g., one antibody, two antibodies, three antibodies, four antibodies, five antibodies, six antibodies, seven antibodies, eight antibodies, nine antibodies, ten antibodies). In some embodiments, the biological sample is stained with six antibodies. In some embodiments, the biological sample is stained with four antibodies. In some embodiments, the biological sample is stained with a second antibody which detects the antibody. In some embodiments, the second antibody is conjugated to a label.

In some embodiments, the label is a detectable label. In some embodiments, the label is a fluorophore. In some embodiments, the detectable label can be directly detectable by itself (e.g., radioisotope labels or fluorescent labels) or, in the case of an enzymatic label, can be indirectly detectable, e.g., by catalyzing chemical alterations of a chemical substrate compound or composition, which chemical substrate compound or composition is directly detectable. In some embodiments, detectable labels can include, but are not limited to, radioisotopes, fluorophores, chemiluminescent compounds, bioluminescent compounds, and dyes.

In some embodiments, the substrate is a slide. In some embodiments, the biological sample comprises a tissue, a tissue section, an organ, an organism, an organoid, or a cell culture sample. In some embodiments, the tissue is a formalin-fixed paraffin-embedded (FFPE) tissue. In some embodiments, the biological sample is fixed prior to the staining step. In some embodiments, the biological sample can be fixed using formalin-fixation and paraffin-embedding (FFPE). In some embodiments, a biological sample can be fixed in any of a variety of other fixatives to preserve the biological structure of the sample prior to analysis. For example, a sample can be fixed via immersion in ethanol, methanol, acetone, formaldehyde (e.g., 2% formaldehyde), paraformaldehyde-Triton, glutaraldehyde, or combinations thereof.

In some embodiments, a compatible fixation method is chosen and/or optimized based on a desired workflow. For example, formaldehyde fixation may be chosen as compatible for workflows using IHC/IF protocols for protein visualization. As another example, methanol fixation may be chosen for workflows emphasizing RNA/DNA library quality. Acetone fixation may be chosen in some applications to permeabilize the tissue. In some embodiments, the biological sample is fixed with formaldehyde. In some embodiments, the biological sample is fixed with methanol.

### Image Analysis and Processing

In some embodiments, the method described herein further includes imaging the biological sample disposed on a substrate, wherein an image of a high-power field (HPF) is generated. As used herein, “high-power field (HPF)” refers to the area of a slide of view under the high magnification power of a microscope. In some embodiments, the imaging step can generate one or more HPFs. In some embodiments, the imaging step can generate up to about 5000 (e.g., about 4500, about 4000, about 3500, about 3000, about 2500, about 2000, about 1500, about 1400, about 1300, about 1200, about 1100, about 1000, about 900, about 800, about 700, about 600, about 500, about 400, about 300, about 200, about 100, about 50, about 40, about 30, about 20, about 10, about 5, about 4, about 3, or about 2) HPFs. In some embodiments, the imaging step includes performing immunofluorescence microscopy on the biological sample.

In some embodiments, provided herein are methods of improving predictive value of a biomarker including: (a) obtaining a plurality of images of a high-power field (HPF) generated from a biological sample; (b) detecting a biomarker in each of the plurality of images; (c) selecting a sub-plurality of images from the plurality of images of step (a); (d) analyzing the sub-plurality of images; and (e) generating an area under the curve value that is greater than an area under the curve value generated when analyzing all of the images, thereby improving predictive value of the biomarker. In some embodiments, a sub-plurality of images can be about 30% (e.g., about 5%, about 10%, about 20%, about 40%, or about 50%) of the plurality of images. In some embodiments, the sub-plurality of images can be up to 100% (e.g., up to 5%, up to 10%, up to 20%, up to 30%, up to 40%, up to 50%, up to 60%, up to 70%, up to 80%, or up to 90%) of the plurality of images.

In some embodiments, the analyzing step of the method described herein can further include (i) image acquisition and processing; (ii) cell segmentation and phenotyping; and (iii) image normalization. Methods for analyzing and processing images of the biological sample are described, for example, in PCT Application No. WO2020/061327 and U.S. patent application Ser. No. 17/278,112, the entire content of each of which are incorporated herein by reference.

In some embodiments, a method may include obtaining, by a device, a plurality of field images of a specimen. In some embodiments, the plurality of field images may be captured by a microscope. In some embodiments, the method may include processing, by the device, the plurality of field images to derive a plurality of processed field images. In some embodiments, the processing may include applying, to the plurality of field images, spatial distortion corrections and illumination-based corrections to address deficiencies in one or more field images of the plurality of field images. In some embodiments, the method may include identifying, by the device and in each processed field image of the plurality of processed field images, a primary area that includes data useful for cell characterization or characterization of subcellular features, identifying, by the device, areas of overlap in the plurality of processed field images, and deriving, by the device, information regarding a spatial mapping of one or more cells of the specimen. In some embodiments, deriving the information may be based on performing, by the device, image segmentation based on the data included in the primary area of each processed field image of the plurality of processed field images, and obtaining, by the device, flux measurements based on other data included in the areas of overlap. In some embodiments, the method may include causing, by the device and based on the information, an action to be performed relating to identifying features related to normal tissue, diagnosis or prognosis of disease, or factors used to select therapy.

In some embodiments, a device may include one or more memories, and one or more processors, communicatively coupled to the one or more memories, configured to obtain a plurality of field images of a tissue sample. In some embodiments, the plurality of field images may be captured by a microscope. In some embodiments, the one or more processors may be configured to apply, to the plurality of field images, spatial distortion corrections and illumination-based corrections to derive a plurality of processed field images, identify, in each processed field image of the plurality of processed field images, a primary area that includes data useful for cell characterization, identify, in the plurality of processed field images, areas that overlap with one another, and derive information regarding a spatial mapping of one or more cells of the tissue sample. In some embodiments, the one or more processors, when deriving the information, may be configured to perform segmentation, on a subcellular level, a cellular level, or a tissue level, based on the data included in the primary area of each processed field image of the plurality of processed field images, and obtain flux measurements based on other data included in the areas that overlap with one another, and cause the information to be loaded in a data structure to enable statistical analysis of the spatial mapping for identifying predictive factors for immunotherapy.

In some embodiments, a non-transitory computer-readable medium may store instructions. In some embodiments, the instructions may include one or more instructions that, when executed by one or more processors, cause the one or more processors to obtain a plurality of field images of a tissue sample, apply, to the plurality of field images, spatial distortion corrections and/or illumination-based corrections to derive a plurality of processed field images, identify, in each processed field image of the plurality of processed field images, a primary area that includes data useful for cell characterization, identify, in the plurality of processed field images, areas that overlap with one another, and derive spatial resolution information concerning one or more cells or subcellular components of the tissue sample. In some embodiments, the one or more instructions, that cause the one or more processors to derive the spatial resolution information, cause the one or more processors to perform image segmentation based on the data included in the primary area of each processed field image of the plurality of processed field images, and obtain flux measurements based on other data included in the areas that overlap with one another. In some embodiments, the one or more instructions, when executed by the one or more processors, may cause the one or more processors to cause a data structure to be populated with the spatial resolution information to enable statistical analyses useful for identifying predictive factors, prognostic factors, or diagnostic factors for one or more diseases or associated therapies.

In some embodiments, the step of image acquisition includes compiling a plurality of HPF images to acquire an image of the whole biological sample within the substrate. In some embodiments, the step of image acquisition includes compiling a plurality of HPF images to acquire an image of a portion of the biological sample. In some embodiments, the plurality of HPF images are from the same tumor. In some embodiments, the plurality of HPF images are from a different tumor. In some embodiments, the plurality of HPF images are generated from the same microscope. In some embodiments, the plurality of HPF images are generated from a different microscope. In some embodiments, the plurality of HPF images are generated from imaging data from scans from chromogenic IHC slides. In some embodiments, the plurality of HPF images are generated from imaging data from tissue-based mass spectrometry. In some embodiments, the plurality of HPF images are generated from imaging data from harvesting spatially-resolved single cells for genomic and transcriptomic analysis. In some embodiments, the plurality of HPF images are sorted/ranked by a feature in an image. In some embodiments, the feature can be the expression of a biomarker. In some embodiments, the feature can be the expression of a CD8 marker. In some embodiments, the feature can be CD163 cells, FoxP3 cells, CD163 PD-L1^{neg} cells, tumor cells, tumorPD-L1+^{mid} cells, FoxP3CD8PD-1+^{low} cells, FoxP3PD-1^{low}+PD-L1+ cells, FoxP3CD8 PD-L1+^{mid} cells, other cells PD-1^{low}+, PDLI+ cells, FoxP3CD8+PD-1-^{mid} cells, CD163 PD-LI+ cells, or any combination thereof.

In some embodiments, the compiling comprises aligning the plurality of HPF images with an overlap. In some embodiments, each HPF image of the plurality of HPF images can overlap an adjacent image by about 20% (e.g., about 5%, about 6%, about 7%, about 8%, about 9%, about 10%, about 11%, about 12%, about 13%, about 14%, about 15%, about 16%, about 17%, about 18%, about 19%, about 21%, about 22%, about 23%, about 24%, about 25%, about 26%, about 27%, about 28%, about 29%, or about 30%). In some embodiments, each HPF image of the plurality of HPF images can overlap an adjacent image by up to 100% (e.g., up to 10%, up to 20%, up to 30%, up to 40%, up to 50%, up to 60%, up to 70%, up to 80%, or up to 90%).

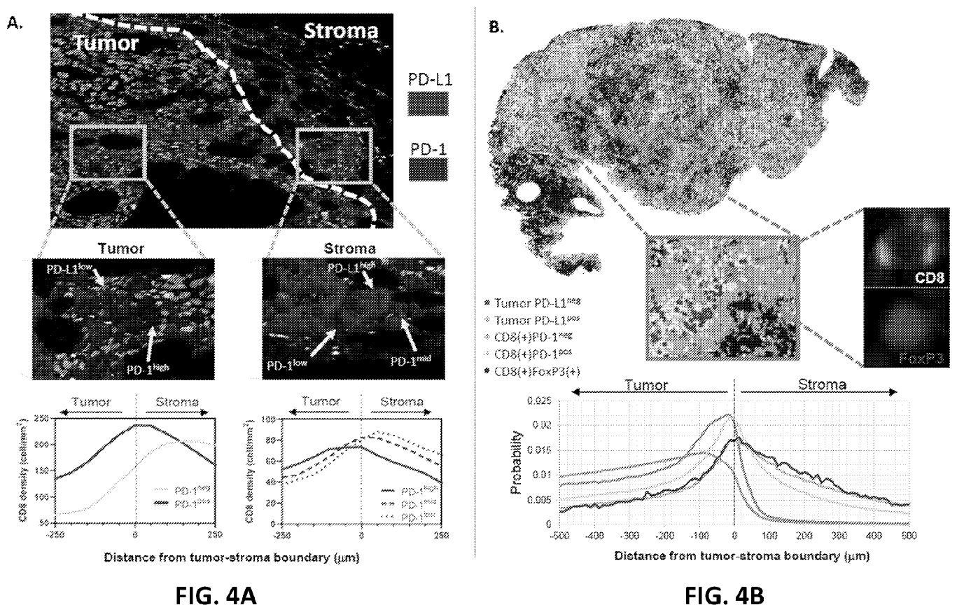

In some embodiments, the step of cell segmentation and phenotyping includes identifying a cell type in the biological sample. In some embodiments, cell segmentation can be performed by delineating membranes of larger cells separate from highlighting smaller lymphocytes. In some embodiments, the step of phenotyping includes detecting expression of at least one of the biomarkers in the cell type. In some embodiments, the expression of at least one biomarker is designated as low, medium, or high. In some embodiments, phenotyping can include detecting expression of a single biomarker, wherein a cell is designated a status of low, medium, or high for the single biomarker. In some embodiments, phenotyping can include detecting expression of multiple biomarkers, wherein individual phenotypes from the single biomarkers are merged to determine cell phenotypes with multiple biomarkers. In some embodiments, phenotyping can include detecting expression of PD-1 expression. In some embodiments, phenotyping can include detecting expression of PD-L1 expression (FIG. 4A). In some embodiments, the cell type comprises a CD163+macrophage, a CD8+ T cell, a Treg cell (CD8negFoxP3+), a tumor cell, a CD8+FoxP3+ cell, or any combinations thereof. In some embodiments, the cell type is determined as negative for a biomarker, wherein the biomarker is not expressed in the cell. In some embodiments, the cell type of CD8+FoxP3+PD-1^{low/mid} is identified as an indicator that the subject will respond to the immunotherapy. In some embodiments, the cell type of CD163+PD-L1^{neg} is identified as an indicator that the subject will not respond to the immunotherapy to the same extent as a reference subject that is identified as not having a cell type of CD163+PD-L1^{neg}.

In some embodiments, the step of cell segmentation and phenotyping further comprises determining a density of the cell type in the biological sample. In some embodiments, the density of the cell type can be used as an indicator of a subject's response to immunotherapy. In some embodiments, a density of total PD-L1+ cells and tumor PD-L1+ cells can be identified as an indicator of response to the immunotherapy. In some embodiments, the density of CD163+PD-L1+ cells does not correlate with a response to immunotherapy. In some embodiments, a high density of CD8+FoxP3+ cells is identified as an indicator that the subject will respond to the immunotherapy.

In some embodiments, the step of image normalization comprises calibrating a fluorescence intensity of at least one of the biomarkers in the plurality of HPF images against a tissue micro array. In some embodiments, image normalization comprises calibrating the fluorescence intensity of PD-1 intensity. In some embodiments, image normalization comprises calibrating the fluorescence intensity of PD-L1 intensity.

In some embodiments, the analyzing step further includes identifying the at least one biomarker in the biological sample from a subject having a disease, and wherein the identification of the at least one biomarker is used to predict the subject's response to immunotherapy.

### Examples

The disclosure is further described in the following examples, which do not limit the scope of the disclosure described in the claims.

### Example 1—Case Selection

Staining optimization of the mIF assays was performed on archival, formalin-fixed paraffin-embedded (FFPE) sections of tonsil and melanoma. Once the index mIF assay (PD-1, PD-L1, CD8, FoxP3, CD163, S100/Sox10) was optimized, a retrospective analysis was performed on a Discovery cohort of pre-treatment FFPE tumor specimens from 53 patients with metastatic melanoma who went on to receive anti-PD-1-based therapy. Thirty-four patients received anti-PD-1 monotherapy (nivolumab or pembrolizumab) and 19 patients received dual anti-PD-1/CTLA-4 blocking therapy (nivolumab and ipilimumab). Patients were classified as responders (complete response or partial response) or non-responders on the basis of RECIST 1.1 criteria. 5-year overall and progression free survival information was also determined. Additional clinicopathologic characteristics of the cohort were also collected, such as age, sex, and stage of disease, Table 1. A single representative FFPE block was chosen for mIF staining. The PD-L1 IHC companion diagnostic assay (22C3) was also performed on these specimens. An independent Validation cohort of pre-treatment FFPE tumor specimens from 45 patients with metastatic melanoma was also studied, Table 2. The optimized 6-plex mIF assay was applied to these specimens and correlated with objective response and long-term survival. Cases in both the Discovery and Validation cohorts were reviewed by a board-certified dermatopathologist to confirm the diagnosis of melanoma. Cases with less than 5 mm of tumor on the slide, those with extensive necrosis or folded tissue, or those of a pure desmoplastic histologic subtype were excluded from analysis.

A separate tissue microarray (TMA) was used to characterize the lymphocyte subsets expressing PD-1 in the melanoma TME, using a second mIF assay (PD-1, CD8, CD4, CD20, FoxP3 and Sox10/S100). The TMA contained tissue from ninety-four patients with metastatic melanoma. A single representative formalin-fixed, paraffin-embedded (FFPE) block from each tumor specimen was chosen for inclusion in the tissue microarray. Six 1.2 mm cores were taken from each block representing both the central and peripheral areas of the tumor and tiled in a tissue microarray format. The resultant TMAs were reviewed, and cores with tissue folds, excessive necrosis, and/or <10% surface area occupied by tumor cells were excluded from analysis.

TABLE 1

| | | | Age at | | [symbol] | Tumor area | OS | OS | PFS | (PFS) | Survival |
|---|---|---|---|---|---|---|---|---|---|---|---|
| Sample ID | Response | Treatment | Collection | Sex | Site | on slide (mm²) | (years) | Status | (years) | Status | Group |
| 1 | Responder | Pembrolizumab | 6 [symbol] | Female | Skin_soft- | 5.811 | 1.680 | Dead | 0.6 [symbol] 0 | Progressor | Poor |
| | | | | | tissue | | | | | | |
| 2 | Responder | Pembrolizumab | 5 [symbol] | Male | Lymph- | 5.566 | 3.953 | Alive | 3.599 | Non- | Good |
| | | | | | node | | | | | Progressor | |
| 3 | Responder | Nivolumab | 7 [symbol] | Female | Skin_soft- | 113.013 | 4.397 | Alive | 2.167 | Progressor | Good |
| | | | | | tissue | | | | | | |
| 4 | Responder | Ipilimumab + | 4 [symbol] | Male | Skin_soft- | 25.608 | 3. [symbol] 0 | Dead | 0.462 | Progressor | Good |
| | | nivolumab | | | tissue | | | | | | |
| 5 | Non- | Ipilimumab + | 78 | Male | Skin_soft- | 95.243 | 3.296 | Dead | 0.250 | Progressor | Poor |
| | Responder | nivolumab | | | tissue | | | | | | |
| 6 | Responder | Ipilimumab + | [symbol] | Female | Skin_soft- | 15.372 | 3.528 | Alive | 3. [symbol] | Non- | Int |
| | | nivolumab | | | tissue | | | | | Progressor | |
| 7 | Responder | Nivolumab | 80 | Male | Skin_soft- | 1 [symbol] .102 | 3.912 | Alive | 4. [symbol] 06 | Non- | [symbol] |
| | | | | | tissue | | | | | Progressor | |
| 8 | Non- | Nivolumab | 67 | Male | [symbol] | 57.358 | 2.015 | Alive | 0.137 | Progressor | Poor |
| | Responder | | | | | | | | | | |
| 9 | Responder | Pembrolizumab | [symbol] | Male | Skin_soft- | 121.292 | 3.562 | Alive | 3.560 | Non- | Good |
| | | | | | tissue | | | | | Progressor | |
| 10 | Responder | Ipilimumab + | [symbol] | Male | [symbol] | 12.4 [symbol] | 2. [symbol] 03 | Alive | 4.917 | Non- | Int |
| | | nivolumab | | | | | | | | Progressor | |
| 11 | Responder | Ipilimumab + | [symbol] | Male | [symbol] | 55.978 | 3.137 | Alive | 4.482 | Non- | Int |
| | | nivolumab | | | | | | | | Progressor | |
| 12 | Responder | Pembrolizumab | 7 [symbol] | Male | [symbol] | 78.560 | 2.485 | Alive | [symbol] .586 | Progressor | Poor |
| 13 | Responder | Nivolumab | [symbol] | Male | Skin_soft- | 38.365 | 9.411 | Alive | 9.408 | Non- | Poor |
| | | | | | tissue | | | | | Progressor | |
| 14 | Responder | Nivolumab | 49 | Male | [symbol] | 32. [symbol] 67 | 9. [symbol] 03 | Alive | 10. [symbol] | Non- | Int |
| | | | | | | | | | | Progressor | |
| 15 | Responder | Ipilimumab + | [symbol] | Female | Lymph- | 56.080 | 2.340 | Alive | 1.020 | Progressor | Good |
| | | nivolumab | | | node | | | | | | |
| 16 | Responder | Pembrolizumab | [symbol] 2 | Male | Lymph- | 25. [symbol] | 3.121 | Alive | 3.855 | Non- | Good |
| | | | | | node | | | | | Progressor | |
| 17 | Responder | Ipilimumab + | [symbol] | Male | Skin_soft- | 205.252 | 4.389 | Alive | 4.755 | Non- | Good |
| | | nivolumab | | | tissue | | | | | Progressor | |
| 18 | Non- | Ipilimumab + | [symbol] | Female | Skin_soft- | 41.004 | 1.164 | Dead | 0.250 | Progressor | Poor |
| | Responder | nivolumab | | | tissue | | | | | | |
| 19 | Responder | Pembrolizumab | 67 | Male | Brain | 309.4 [symbol] 0 | 3.370 | Alive | 3.7 [symbol] 4 | Non- | Good |
| | | | | | | | | | | Progressor | |
| 20 | Responder | Pembrolizumab | 68 | Female | Skin_soft- | 60. [symbol] 48 | 2.284 | Dead | 0.333 | Progressor | Int |
| | | | | | tissue | | | | | | |
| 21 | Non- | Pembrolizumab | [symbol] 0 | Female | Skin_soft- | 14.054 | 1.04 [symbol] | Dead | 0.218 | Progressor | Good |
| | Responder | | | | tissue | | | | | | |
| 22 | Non- | Ipilimumab + | [symbol] | Male | [symbol] | 197.026 | 1.030 | Dead | 0. [symbol] | Progressor | Poor |
| | Responder | nivolumab | | | | | | | | | |
| 23 | Responder | Pembrolizumab | 63 | Female | Skin_soft- | 23.209 | 1.877 | Alive | 1.13 [symbol] | Progressor | Int |
| | | | | | tissue | | | | | | |
| 24 | Responder | Ipilimumab + | 59 | Female | Skin_soft- | 31.925 | 2.014 | Alive | 2.2 [symbol] 4 | Non- | Good |
| | | nivolumab | | | tissue | | | | | Progressor | |
| 25 | Non- | Pembrolizumab | 76 | Male | Lymph- | 41.25 | 1.0 [symbol] 3 | Dead | 0.105 | Progressor | Int |
| | Responder | | | | node | | | | | | |
| 26 | Responder | Pembrolizumab | 82 | Male | Skin_soft- | 94.481 | 1. [symbol] 03 | Dead | 0.183 | Non- | Int |
| | | | | | tissue | | | | | Progressor | |
| 27 | Responder | Nivolumab | 74 | Female | Skin_soft- | 14.975 | 2.321 | Alive | 0.685 | Progressor | Good |
| | | | | | tissue | | | | | | |
| 28 | Responder | Pembrolizumab | 53 | Male | Skin_soft- | 35.623 | 2.119 | Alive | 2.411 | Non- | Good |
| | | | | | tissue | | | | | Responder | |
| 29 | Non- | Pembrolizumab | 83 | Female | Lymph- | 13.0 [symbol] 2 | 1.608 | Alive | 0.191 | Progressor | Int |
| | Responder | | | | node | | | | | | |
| 30 | Non- | Pembrolizumab | 67 | Female | [symbol] | 7. [symbol] 7 | 1.688 | Alive | 0.110 | Progressor | Poor |
| | Responder | | | | | | | | | | |
| 31 | Responder | Ipilimumab + | [symbol] | Female | Skin_soft- | 14.320 | 4.485 | Alive | 5.355 | Non- | Good |
| | | nivolumab | | | tissue | | | | | Responder | |
| 32 | Non- | Pembrolizumab | [symbol] | Female | Lymph- | 217. [symbol] 77 | 1.249 | Dead | 0.382 | Progressor | Poor |
| | Responder | | | | node | | | | | | |
| 33 | Responder | Nivolumab | 76 | Male | Skin_soft- | 202.768 | 2.819 | Alive | 3.310 | Non- | Good |
| | | | | | tissue | | | | | Responder | |
| 34 | Responder | Nivolumab | 53 | Female | Lymph- | 7.9 [symbol] 1 | 3.352 | Dead | 1.887 | Progressor | Int |
| | | | | | node | | | | | | |
| 35 | Responder | Pembrolizumab | 73 | Male | Skin_soft- | 35. [symbol] 02 | 1.978 | Alive | 2. [symbol] | Non- | Int |
| | | | | | tissue | | | | | Responder | |
| 36 | Non- | Nivolumab | [symbol] | Male | [symbol] | 37. [symbol] | [symbol] | Dead | 0.460 | Progressor | Poor |
| | Responder | | | | | | | | | | |
| 37 | Responder | Ipilimumab + | 73 | Female | [symbol] | 30 [symbol] .768 | 3. [symbol] 23 | Alive | 4.152 | Non- | Int |
| | | nivolumab | | | | | | | | Responder | |
| 38 | Non- | Pembrolizumab | [symbol] 2 | Female | [symbol] | 38.304 | 1.482 | Alive | 0.108 | Progressor | Int |
| | Responder | | | | | | | | | | |
| 39 | Responder | Ipilimumab + | [symbol] 2 | Male | Skin_soft- | [symbol] 1.1 [symbol] | 2.891 | Dead | 2.086 | Non- | Poor |
| | | nivolumab | | | tissue | | | | | Responder | |
| 40 | Responder | Ipilimumab + | [symbol] | Male | Skin_soft- | 74.265 | 4.872 | Alive | 5.335 | Non- | Good |
| | | nivolumab | | | tissue | | | | | Responder | |
| 41 | Responder | Ipilimumab + | [symbol] | Female | Lymph- | 36.876 | 3.104 | Dead | 0.733 | Progressor | Int |
| | | nivolumab | | | node | | | | | | |
| 42 | Responder | Ipilimumab + | [symbol] | Male | Skin_soft- | 102.75 [symbol] | 1.961 | Alive | 2. [symbol] 45 | Non- | Int |
| | | nivolumab | | | tissue | | | | | Responder | |
| 43 | Non- | Nivolumab | [symbol] | Female | Skin_soft- | 27.733 | 2.605 | Alive | 0.417 | Progressor | Poor |
| | Responder | | | | tissue | | | | | | |
| 44 | Non- | Pembrolizumab | 33 | Male | Skin_soft- | 25.48 [symbol] | 2.630 | Dead | 1.753 | Progressor | Poor |
| | Responder | | | | tissue | | | | | | |
| 45 | Non- | Nivolumab | [symbol] | Female | Skin_soft- | 1 [symbol] .466 | 1.000 | Alive | 0.228 | Progressor | Poor |
| | Responder | | | | tissue | | | | | | |
| 46 | Non- | Nivolumab | [symbol] | Male | Lymph- | [symbol] .472 | 1.485 | Dead | 0.419 | Progressor | Poor |
| | Responder | | | | node | | | | | | |
| 47 | Non- | Nivolumab | 63 | Male | Skin_soft- | 3 [symbol] .768 | 1.634 | Alive | 0.357 | Progressor | Poor |
| | Responder | | | | tissue | | | | | | |
| 48 | Non- | Ipilimumab + | 35 | Female | [symbol] | 6. [symbol] 65 | 0.7 [symbol] 2 | Dead | 0.218 | Progressor | Poor |
| | Responder | nivolumab | | | | | | | | | |
| 49 | Responder | Ipilimumab + | 51 | Female | Skin_soft- | [symbol] 3.867 | 2.085 | Alive | 1.433 | Progressor | Int |
| | | nivolumab | | | tissue | | | | | | |
| 50 | Non- | Nivolumablumab | 72 | Male | Skin_soft- | 52.684 | 1.911 | Dead | 0. [symbol] | Progressor | Int |
| | Responder | | | | tissue | | | | | | |
| 51 | Non- | Ipilimumab + | [symbol] | Female | [symbol] | 43.078 | 0.25 [symbol] | Dead | 0.250 | Progressor | Poor |
| | Responder | nivolumab | | | | | | | | | |
| 52 | Responder | Pembrolizumab | [symbol] | Female | Skin_soft- | 8.732 | 1.836 | Alive | 1. [symbol] | Progressor | Good |
| | | | | | tissue | | | | | | |
| 53 | Responder | Pembrolizumab | 75 | Female | Skin_soft- | 75.748 | 1.582 | Alive | 1. [symbol] | Non- | Poor |
| | | | | | tissue | | | | | Responder | |

[symbol]

indicates data missing or illegible when filed

TABLE 2

| | | | Age at | | Tumor area | OS | OS | PFS | (PFS) | Survival |
|---|---|---|---|---|---|---|---|---|---|---|
| Sample ID | Response | Treatment | Collection | Sex | on slide (mm²) | (years) | Status | (years) | Status | Group |
| 1 | Non- | Ipilimumab + | 72 | Male | 121.292 | 3.712 | Alive | 0.134 | Progressor | Poor |
| | Responder | nivolumab | | | | | | | | |
| 2 | Responder | Pembrolizumab | 67 | Female | 113.011 | 2.897 | Alive | 1.814 | Progressor | Poor |
| 3 | Non- | Ipilimumab + | 60 | Female | 60.048 | 2.545 | Alive | 0.173 | Progressor | Int |
| | Responder | nivolumab | | | | | | | | |
| 4 | Responder | Pembrolizumab | 55 | Male | 102.751 | 3.277 | Alive | 3.274 | Non- | Good |
| | | | | | | | | | Progressor | |
| 5 | Responder | Nivolumab | 63 | Male | 309.440 | 2.496 | Alive | 0.844 | Progressor | Poor |
| 6 | Responder | Nivolumab | 44 | Male | 41.026 | 3.710 | Alive | 2.581 | Progressor | Int |
| 7 | Responder | Ipilimumab + | 75 | Male | 38.366 | 2. [symbol] 8 | Alive | 2. [symbol] 2 | Non- | Poor |
| | | nivolumab | | | | | | | Progressor | |
| 8 | Non- | Ipilimumab + | 74 | Male | 11.493 | 3.222 | Dead | 0.222 | Progressor | Poor |
| | Responder | nivolumab | | | | | | | | |
| 9 | Non- | Ipilimumab + | 28 | Female | 55.080 | 0.811 | Dead | 0.205 | Progressor | Poor |
| | Responder | nivolumab | | | | | | | | |
| 10 | Responder | Ipilimumab + | 51 | Male | 94.481 | 2.627 | Alive | 2. [symbol] 59 | Non- | Int |
| | | nivolumab | | | | | | | Progressor | |
| 11 | Responder | Nivolumab | 60 | Male | 74.266 | 2.345 | Dead | 1.162 | Progressor | Good |
| 12 | Non- | Pembrolizumab | 75 | Female | 15.372 | 0.351 | Dead | 0.167 | Progressor | Poor |
| | Responder | | | | | | | | | |
| 13 | Non- | Nivolumab | 69 | Male | 31.925 | 0.638 | Dead | 0.145 | Progressor | Int |
| | Responder | | | | | | | | | |
| 14 | Non- | Ipilimumab + | 61 | Female | 32.767 | 0.715 | Dead | 0.156 | Progressor | Poor |
| | Responder | nivolumab | | | | | | | | |
| 15 | Non- | Ipilimumab + | 77 | Female | 38.304 | 0.2 [symbol] | Dead | 0.129 | Progressor | Good |
| | Responder | nivolumab | | | | | | | | |
| 16 | Responder | Ipilimumab + | 36 | Female | 37.062 | 1.573 | Alive | 2.403 | Non- | Good |
| | | nivolumab | | | | | | | Progressor | |
| 17 | Responder | Pembrolizumab | 73 | Male | 23.484 | 1.137 | Alive | 0.997 | Non- | Good |
| | | | | | | | | | Progressor | |
| 18 | Responder | Ipilimumab + | 68 | Male | 151.159 | 2.337 | Alive | 2.079 | Non- | Good |
| | | nivolumab | | | | | | | Progressor | |
| 19 | Non- | Pembrolizumab | 88 | Male | 17.739 | 1.304 | Alive | 0.197 | Progressor | Good |
| | Responder | | | | | | | | | |
| 20 | Non- | Pembrolizumab | 86 | Female | 13.209 | 0.967 | Dead | 0.170 | Progressor | Int |
| | Responder | | | | | | | | | |
| 21 | Non- | Nivolumab | [symbol] 0 | Male | 6.811 | 0.62 [symbol] | Dead | 0.211 | Progressor | Poor |
| | Responder | | | | | | | | | |
| 22 | Responder | Ipilimumab + | 49 | Female | 25.608 | 2.781 | Alive | 2.762 | Non- | Poor |
| | | nivolumab | | | | | | | Progressor | |
| 23 | Non- | Ipilimumab + | 60 | Male | 14.075 | 1.655 | Dead | 0.181 | Progressor | Int |
| | Responder | nivolumab | | | | | | | | |
| 24 | Responder | Ipilimumab + | 56 | Female | 55.978 | 1.652 | Alive | 0.510 | Progressor | Poor |
| | | nivolumab | | | | | | | | |
| 25 | Responder | Ipilimumab + | 62 | Female | 35.623 | 4.984 | Alive | 0.518 | Progressor | Int |
| | | nivolumab | | | | | | | | |
| 26 | Responder | Pembrolizumab | 64 | Female | 14.464 | 3.567 | Alive | 3.562 | Non- | Int |
| | | | | | | | | | Progressor | |
| 27 | Non- | Pembrolizumab | 74 | Male | 15.002 | 1.222 | Alive | 0.219 | Progressor | Good |
| | Responder | | | | | | | | | |
| 28 | Responder | Pembrolizumab | 40 | Female | 25.919 | 2.205 | Alive | 1.279 | Progressor | Poor |
| 29 | Non- | Nivolumab | 67 | Male | 57.953 | 0.066 | Dead | 0.041 | Progressor | Poor |
| | Responder | | | | | | | | | |
| 30 | Non- | Nivolumab | [symbol] 6 | Male | 41.004 | 0.8 [symbol] | Dead | 0.044 | Progressor | Poor |
| | Responder | | | | | | | | | |
| 31 | Non- | Pembrolizumab | [symbol] 2 | Female | 13.061 | 2.858 | Dead | 0.219 | Progressor | Int |
| | Responder | | | | | | | | | |
| 32 | Responder | Pembrolizumab | 52 | Male | 197.926 | 3.203 | Alive | 3.019 | Non- | Int |
| | | | | | | | | | Progressor | |
| 33 | Responder | Nivolumab | 69 | Female | 7.997 | 1.397 | Dead | 0.595 | Progressor | Int |
| 34 | Non- | Ipilimumab + | 59 | Male | 78.560 | 0.652 | Dead | 0.099 | Progressor | Poor |
| | Responder | nivolumab | | | | | | | | |
| 35 | Non- | Nivolumablumab | 82 | Male | 15.102 | 0.548 | Dead | 0.170 | Progressor | Poor |
| | Responder | | | | | | | | | |
| 36 | Responder | Ipilimumab + | 52 | Male | 206.252 | 5.471 | Alive | 5.425 | Non- | Poor |
| | | nivolumab | | | | | | | Progressor | |
| 37 | Responder | Pembrolizumab | 71 | Female | 73.748 | 0.9 [symbol] 9 | Alive | 0.740 | Non- | Good |
| | | | | | | | | | Progressor | |
| 38 | Responder | Pembrolizumab | 85 | Male | 309.208 | 2.512 | Alive | 2.510 | Non- | Good |
| | | | | | | | | | Progressor | |
| 39 | Responder | Ipilimumab + | 69 | Female | 14.320 | 4.260 | Alive | 4.252 | Non- | Int |
| | | nivolumab | | | | | | | Progressor | |
| 40 | Responder | Ipilimumab + | 64 | Male | 36.876 | 5.178 | Alive | 5.170 | Non- | Good |
| | | nivolumab | | | | | | | Progressor | |
| 41 | Responder | Nivolumab | 34 | Male | 202.748 | 2.742 | Alive | 1.115 | Progressor | Good |
| 42 | Non- | Ipilimumab + | 56 | Female | 95.243 | 1.033 | Dead | 0.233 | Progressor | Poor |
| | Responder | nivolumab | | | | | | | | |
| 43 | Responder | Pembrolizumab | 63 | Male | 217.077 | 1.885 | Alive | 1.877 | Non- | Int |
| | | | | | | | | | Progressor | |
| 44 | Responder | Pembrolizumablizumab | 52 | Male | 5.560 | 2.378 | Alive | 2.282 | Non- | Poor |
| | | | | | | | | | Progressor | |
| 45 | Responder | Ipilimumab + | 56 | Male | 7.961 | 5.101 | Alive | 4.95 [symbol] | Non- | Good |
| | | nivolumab | | | | | | | Progressor | |

[symbol]

indicates data missing or illegible when filed

### Example 2—Reagents and Multispectral Microscope

### Fluorophore Reagents and Multiplex Staining

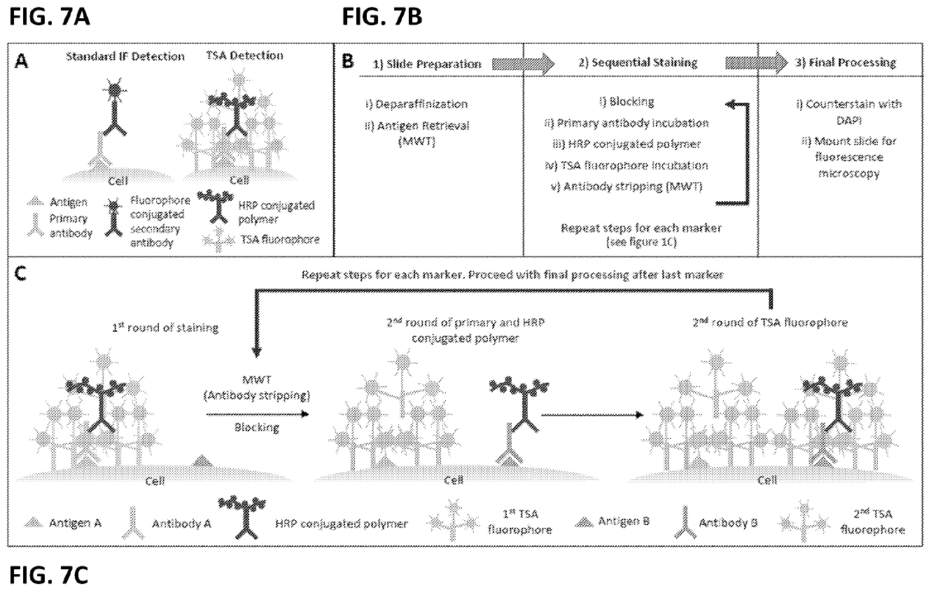

FFPE slides were stained using tyramide signal amplification (TSA) technology in order to achieve superior amplification and higher plexing compared to standard IF detection, FIGS. 7A-7C. In comparison to detection of primary antibodies with directly labeled secondary antibodies, TSA technology utilizes HRP-polymer secondary mediated detection. A single HRP-polymer secondary can catalyze the activation of several fluorophore labeled tyramides (TSA fluorophore). Following activation, the TSA fluorophores can covalently bind to surrounding tyrosine residues and remain deposited on the tissue during heat treatment steps that strip off primary and secondary antibodies. By employing sequential rounds of staining and stripping, we labeled 6 markers plus DAPI on a single FFPE tissue section.

### Slide Scanning and Multispectral Unmixing

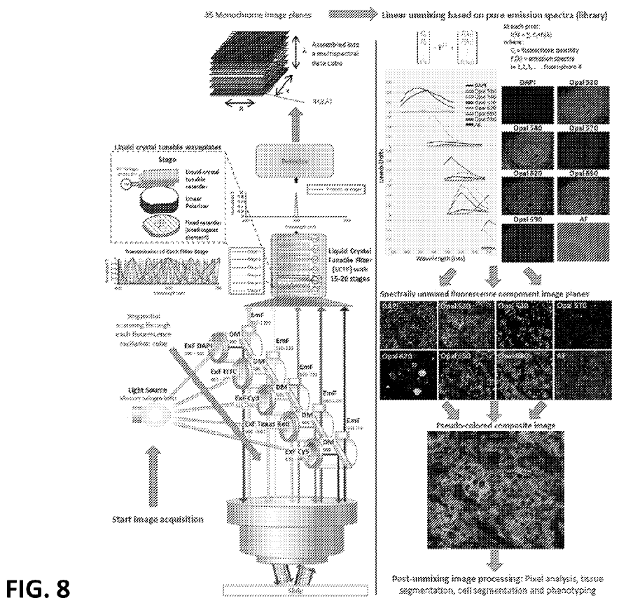

Images were scanned with the Vectra 3.0 Automated Quantitative Pathology Imaging System (Akoya Biosciences) and processed using digital image analysis software, inForm (Ver 2.3, Akoya Biosciences). A schematic of the multispectral imaging microscope system is shown in FIG. 8. The system captures 20X multispectral images consisting of a multilayer image ‘cube’ of 35 image planes. These planes correspond to the wavelengths selected by the liquid crystal tunable filter, acquired across the visible light spectrum. Images of multiplex stained samples are then unmixed, using an inverse least squares fitting approach that minimizes the square difference between the measured and the characteristic emission spectrum of each fluorophore. Unmixing separates the autofluoresence and the overlapping emission signals of each fluorophore, thus removing autofluoresence background and creating eight signal specific ‘component’ planes; one for each fluorophore plus DAPI and autofluoresence.

In order to unmix the multispectral image cube, the known characteristic emission spectra of the TSA fluorophores, DAPI, and a spectrum representative of the background autofluoresence are used to generate an unmixing library. To acquire the pure spectra for the library, 4 μm thick FFPE tonsil sections were stained with anti-CD20 (dilution 1:400, clone L26 Leica microsystems) by monoplex IF (see Monoplex IF section) with each fluorophore. The TSA concentrations were adjusted to obtain pixel normalized fluorescence intensity (NFI) counts of to 15 for each TSA fluorophore (520 1:150, 540 1:500, 570 1:200, 620 1:150, 650 1:200, 690 1:50). DAPI was not added at the end of the protocol. One tonsil section was stained with DAPI alone to extract the DAPI spectrum while the autofluorescence spectrum was extracted from an unstained slide of the tissue of interest. The slides were imaged and the spectra extracted in inForm using automated tools for library creation. Similarly, for spectral unmixing of chromogenic stains, a spectral library of DAB and hematoxylin was used.

### Example 3—Staining Optimization

### Characterizing TSA Fluorophores—Staining Index (SI), Bleed-Through (BT) and Marker Pairing

To explore fluorophore staining indices, sequential slides from five archival tonsil specimens were stained by monoplex IF with anti-CD8 (dilution 1:100, clone 4B11) and each TSA fluorophore at dilution 1:50. Single-cell data was exported from inForm. The SI was calculated as the difference between the mean florescence intensity of the positive and negative cell populations divided by two standard deviations of the negative population.

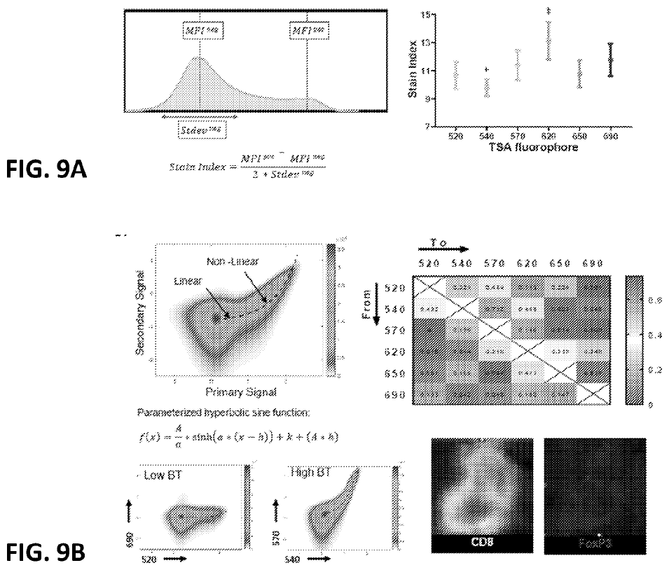

The same tonsil specimens were used to characterize bleed-through or spillover of fluorophore emission spectra, a frequent limitation of multiparametric fluorescent methods. Pairwise dot plots of the logarithm of normalized fluorescence intensity counts were created for all channels. We consistently observed a linear relationship at low intensity counts and an exponential relationship at high intensities, FIGS. 9A-9B. In order to account for this duality a hyperbolic sine curve was parameterized and fit to each paired dataset using a non-linear least squares model. To improve the accuracy of the fit outliers in the noise population were removed. The data was then inverted and centered about the median of the original noise. The propensity of BT was then calculated as the linear term * the non-linear term of the fitted curve.

### Chromogenic Staining

Four-micron thick sections were stained individually for CD8, CD163, PD-1, PD-L1, FoxP3, Sox10, S100 and a Sox10/S100 cocktail. Briefly, slides were deparaffinized, rehydrated, and subjected to heat-induced epitope retrieval (HIER) in pH 6 target antigen retrieval buffer (S1699, Dako) for 10 min at 120° C. (Decloaking chamber, Biocare Medical). Blocking for endogenous peroxidase (3% H2O2, H325-500, Fisher Scientific) and protein (ACE Block, BUF029, Bio-Rad) was performed. For the protocols using a biotinylated secondary antibody, endogenous biotin was also blocked (Avidin/Biotin Blocking Kit, SP-2001, Vector Labs). Primary antibodies were incubated at 4° C. for 22 hrs, followed by secondary antibodies at room temperature (RT) for 30 min, as noted in Table 3. For the protocols using a biotinylated secondary antibody, a tyramide signal amplification (TSA) system was used as described previously. Antigen-antibody binding was visualized with the use of 3,3′-diaminobenzidine (D4293, Sigma). Slides were counterstained with hematoxylin and coverslipped (VectaMount, H-5000, VectorLabs).

TABLE 3

| | Primary Antibody | | | | Secondary Antibody | | |
|---|---|---|---|---|---|---|---|
| Marker | Species | Clone | Source | Final [μg/mL) | Amplification | Source | Final [μg/mL] |
| FoxP3 | Mouse | 236A/E7 | Affymetrix | 5.00 | Anti-Mouse | MP-7402, | RTU |
| | | | | | Polymer HRP | Vector Labs | |
| CD8 | Mouse | 4811 | AbD | * | Anti Mouse | MP-7402, | RTG |
| | | | Seroton | | Polymer ARP | Vector Labs | |
| Sox10 | Mouse | BC34 | BioCare | 0.24 | Anti-Mouse | MP-7402, | RTU |
| | | | Medical | | Polymer HRP | Vector tabs | |
| S100 | Mouse | 4C4.9 | Abnova | 0.33 | Anti-Mouse | MP-7402, | RTU |
| | | | | | Polymer HRP | Vector Labs | |
| PD-1 | Mouse | NAT105 | Abcam | 1.00 | Anti-Mouse | 553441, | 1.00 |
| | | | | | Biotinylated | 3D Pharmigen | |
| PD-L1 | Rabbit | SP142 | Spring | 0.10 | Anti Rabbit | 330338, | 1.00 |
| | | | Bioscience | | Biotinylated | 80 Pharmingen | |
| CD163 | Mouse | 10D6 | Leica | 0.49 | Anti-Mouse | MP-7402, | RTU |
| | | | Biosystems | | Polymer HRP | Vector Labs | |

### Monoplex IF

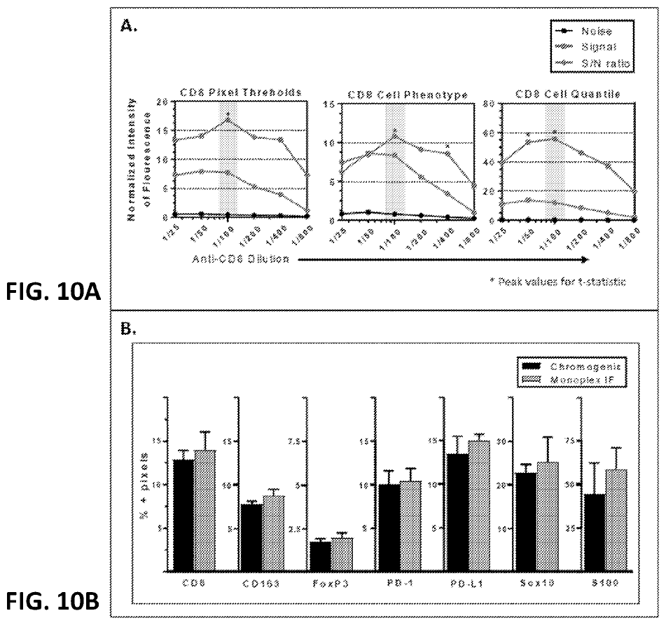

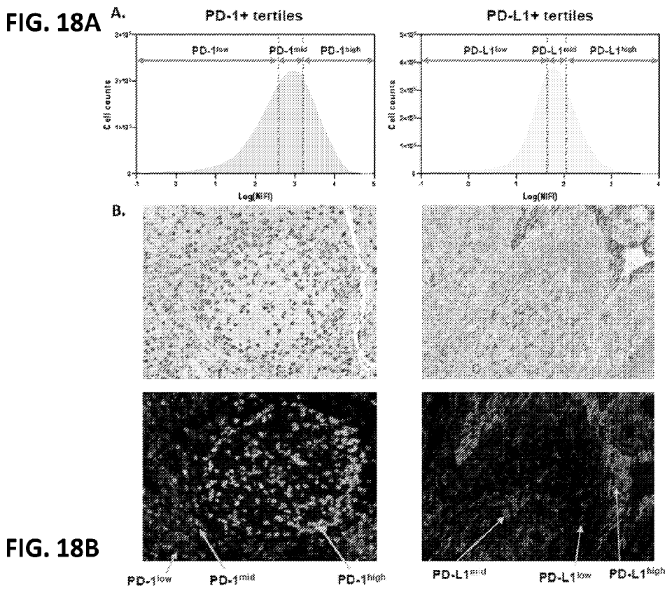

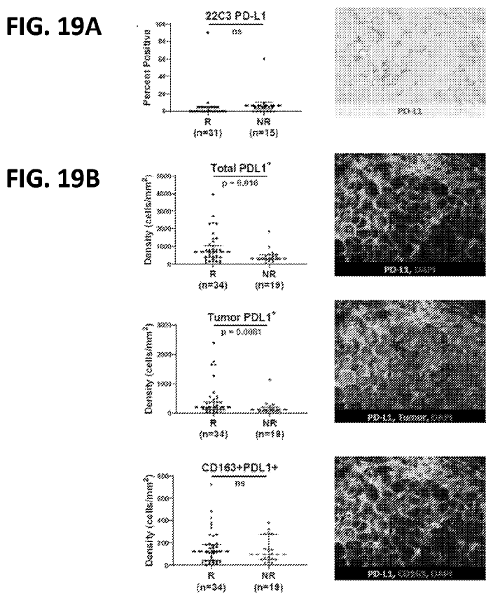

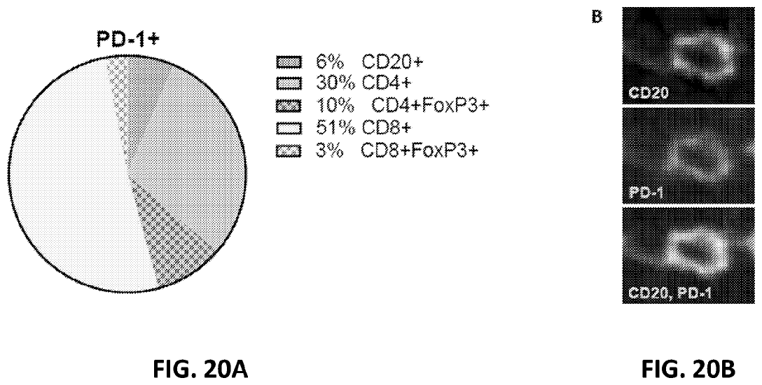

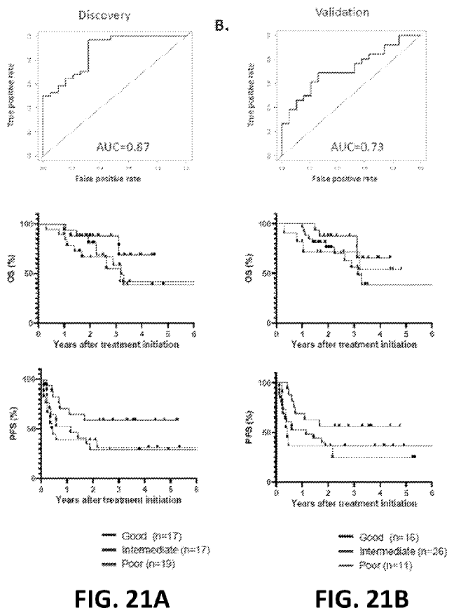

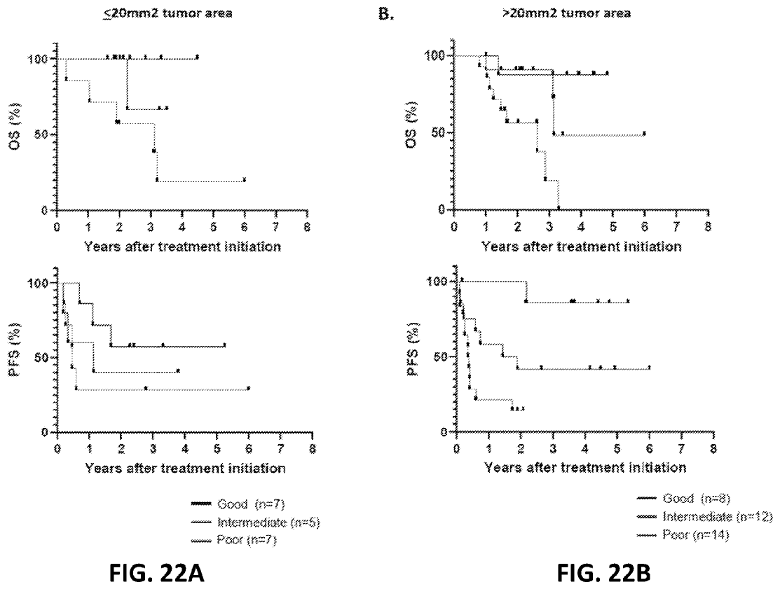

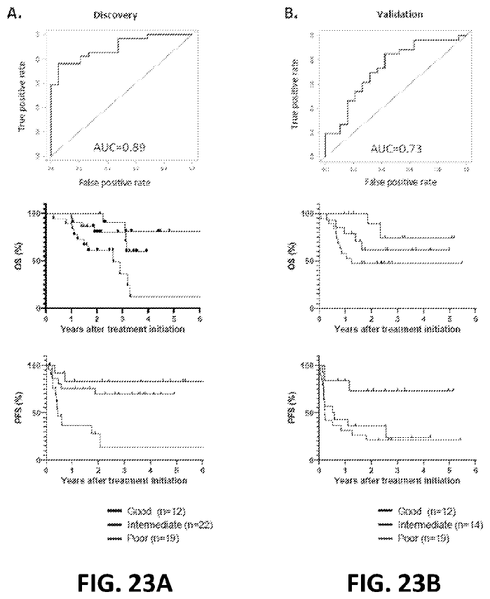

Monoplex IF staining was performed on sequential slides, 3 tonsil and melanoma (for Sox10 and $100), to titrate each primary antibody, Table 4. Briefly, slides were deparaffinized and subjected to microwave HIER (Haier 1000W) in pH 9 followed by pH 6 buffer (AR900 and AR600, respectively, Akoya Biosciences) for 45 sec at 100% power and 15 minutes at 20% power. Endogenous peroxidase removal (3% H2O2, H325-500, Fisher) and protein blocking (Antibody Diluent Background Reducing, S3022, Dako) were performed followed by primary antibody incubations at RT, starting at double the optimal concentration used for chromogenic staining and serially diluting. All secondary antibodies were incubated for 10 min at RT. The TSA fluorophore (Opal 7 color kit, NEL811001KT, Akoya Biosciences) paired with a given marker was then applied for 10 min. A final microwave step was performed at pH 6, slides were stained with DAPI (Opal 7 color kit, NEL811001KT, Akoya Biosciences) and coverslipped (ProLong Diamond Antifade Mountant, P36970, Life Technologies). For comparison of primary titrations 10 corresponding high power fields (HPFs) were selected for each dilution and the signal to noise ratio (SNR) was evaluated using both pixel-based and cell-based approaches (FIG. 10A). Ten corresponding HPFs were also chosen for comparison to chromogenic IHC. HPFs were specifically chosen to capture a broad dynamic range of PD-1 and PD-L1 expression, see FIGS. 18A-18B.

TABLE 4

| | Primary Antibody | | | | | Secondary Antibody | | | | |
|---|---|---|---|---|---|---|---|---|---|---|
| | | | | Final | Incubation | | | Dilution | TSA Fluorophone | |
| Marker | Species | Clone | Source | (μg/mL) | times (min) | Amplification | Source | (In PBS) | [symbol] | Dilution |
| FoxP3 | Mouse | 236A/E7 | Affymetrix | 0.3 [symbol] - [symbol] | 30 | Anti-Rabbit | [symbol] | [symbol] | 570 | 1:50 |
| | | | | | | PowerVision | | | | |
| | | | | | | Poly-HRP | | | | |
| CD8 | Mouse | 4811 | AbD | * | 30 | Opal Polymer HRP | [symbol] | RTU | 540 | 1:50 |
| | | | Seroton | | | Ms + [symbol] | | | | |
| Sox10 | Mouse | BC34 | BioCare | 0.01-0.25 | 60 | Opal Polymer HRP | [symbol] | RTU | 670 | 1:50 |
| | | | Medical | | | Ms + [symbol] | | | | |
| S100 | Mouse | 4C4.9 | Abnova | 0.01-1.00 | | | | | | |
| PD-1 | Rabbit | [symbol] | Abcam | 0.06-7. [symbol] | 30 | Anti-Rabbit | [symbol] | [symbol] | [symbol] 50 | 1:50 |
| | | | | | | PowerVision | | | | |
| | | | | | | Poly-HRP | | | | |
| PD-L1 | Rabbit | SP142 | Spring | 0.0 [symbol] -3.5 [symbol] | 60 | Anti-Rabbit | [symbol] | [symbol] | 520 | [symbol] |
| | | | Bioscience | | | PowerVision | | | | |
| | | | | | | Poly-HRP | | | | |
| CD163 | Mouse | 10D6 | Leica | 0.06- [symbol] | [symbol] | Opal Polymer HRP | [symbol] | RTU | [symbol] | [symbol] |
| | | | Biosystems | | | Ms + [symbol] | | | | |

[symbol]

indicates data missing or illegible when filed

After the optimal primary antibody concentration was identified, TSA titrations were performed on 5 melanoma tumor sections for all markers, Table 5. HIER steps were performed both before and after staining in accordance with how the slides would be treated in the final multiplex assay. Ten corresponding HPFs for each IF condition and the related chromogenic IHC were selected for analysis. Equivalence of signal compared to chromogenic IHC and bleed-through between fluorescent channels was considered to select the optimal TSA concentration for each marker.

TABLE 5

| | [symbol] | | Primary Antibody | | | | | Secondary Antibody | | | | |
|---|---|---|---|---|---|---|---|---|---|---|---|---|
| | Before | After | | | | Final | Incubation | | | Dilution | TSA Fluorophone | |
| Marker | [symbol] | [symbol] | Species | Clone | Source | (μg/mL) | times (min) | Amplification | Source | (in PBS) | Opal | Dilution |
| FoxP3 | [symbol] | [symbol] | Mouse | 236A/E7 | Affymetrix | [symbol] | 30 | Anti-Rabbit | [symbol] | [symbol] | [symbol] | [symbol] |
| | | | | | | | | PowerVision | | | | |
| | | | | | | | | Poly-HRP | | | | |
| CD8 | [symbol] | [symbol] | Mouse | 4811 | AbD | * | 30 | Opal Polymer HRP | [symbol] | RTU | [symbol] | [symbol] |
| | | | | | Seroton | | | Ms + [symbol] | | | | |
| Sox10 | [symbol] | [symbol] | Mouse | BC34 | BioCare | [symbol] | 60 | Opal Polymer HRP | [symbol] | RTU | [symbol] | [symbol] |
| | | | | | Medical | | | Ms + [symbol] | | | | |
| S100 | | | Mouse | 4C4.9 | Abnova | [symbol] | | | | | | |
| PD-1 | [symbol] | [symbol] | Rabbit | [symbol] | Abcam | [symbol] | 30 | Anti-Rabbit | [symbol] | [symbol] | [symbol] | [symbol] |
| | | | | | | | | PowerVision | | | | |
| | | | | | | | | Poly-HRP | | | | |
| PD-L1 | [symbol] | [symbol] | Rabbit | SP142 | Spring | [symbol] | 60 | Anti-Rabbit | [symbol] | [symbol] | [symbol] | [symbol] |
| | | | | | Bioscience | | | PowerVision | | | | |
| | | | | | | | | Poly-HRP | | | | |
| CD163 | [symbol] | [symbol] | Mouse | 10D6 | Leica | [symbol] | [symbol] | Opal Polymer HRP | [symbol] | RTU | [symbol] | [symbol] |
| | | | | | Biosystems | | | Ms + [symbol] | | | | |

[symbol]

indicates data missing or illegible when filed

### Multiplex IF

Single sections from five FFPE melanoma specimens were stained for all 6 markers in the multiplex panel, Table 6. In addition, the three 4 μm thick tissue sections before and after the slide used for the 6-plex panel were stained for the individual markers. Ten HPFs were compared between the multiplex IF and the corresponding monoplex IF.

TABLE 6

| | | Primary Antibody | | | | | Secondary Antibody | | | TSA Fluorophone | |
|---|---|---|---|---|---|---|---|---|---|---|---|
| | | | | | Final | Incubation | | | Dilution | | TSA |
| Position | Marker | Species | Clone | Source | (μg/mL) | times (min) | Amplification | Source | (in PBS) | Opal | dilution |
| 1 | FoxP3 | Mouse | 236A/E7 | Affymetrix | 5.05 | 30 | Anti-Rabbit | [symbol] | [symbol] | [symbol] | 1:200 |
| | | | | | | | PowerVision | | | | |
| | | | | | | | Poly-HRP | | | | |
| 2 | CD8 | Mouse | 4811 | AbD | * | 30 | Opal Polymer HRP | [symbol] | RTU | [symbol] | 1:200 |
| | | | | Seroton | | | Ms + [symbol] | | | | |
| 3 | Sox10 | Mouse | BC34 | BioCare | 0.13 | 60 | Opal Polymer HRP | [symbol] | RTU | [symbol] | 1: [symbol] |
| | | | | Medical | | | Ms + [symbol] | | | | |
| | S100 | Mouse | 4C4.9 | Abnova | 0.06 | | | | | | |
| 4 | PD-1 | Rabbit | [symbol] | Abcam | 0.50 | 30 | Anti-Rabbit | [symbol] | [symbol] | [symbol] | 1:300 |
| | | | | | | | PowerVision | | | | |
| | | | | | | | Poly-HRP | | | | |
| 5 | PD-L1 | Rabbit | SP142 | Spring | 0.1 [symbol] | 60 | Anti-Rabbit | [symbol] | [symbol] | [symbol] | 1:300 |
| | | | | Bioscience | | | PowerVision | | | | |
| | | | | | | | Poly-HRP | | | | |
| 6 | CD163 | Mouse | 10D6 | Leica | [symbol] | [symbol] | Opal Polymer HRP | [symbol] | RTU | [symbol] | 3:50 |
| | | | | Biosystems | | | Ms + [symbol] | | | | |

[symbol]

indicates data missing or illegible when filed

### Approaches to Signal Quantification

Signal was quantified by a number of different approaches, including cell-based and pixel-based approaches, both with and without machine learning. The cell-based approach combined with machine learning is recommended by the manufacturer. It labels individual cell types and assigns them Cartesian coordinates and thus facilitates analysis of cell densities, fluorescence intensities of markers in different cell compartments, marker co-expression, and distance metrics between cells. Cell-based quantification was performed by using the Cell Segmentation Module (which identifies and maps individual cells) in the inForm software, followed by machine-learning based-phenotyping, i.e., assigning a cell-type.

A cell-based approach without machine learning was also used to quantify signal, since it is faster and requires less user input. The Cell Segmentation Module was used to output the mean fluorescence intensity for each fluorophore in the compartment of interest for each cell. The data was then binned into 10% relative intensity intervals, and the median of the top 10% was extracted as signal and the bottom 10% as noise for quantile-based cell analysis.

The pixel-based approaches are not dependent on cell identification, i.e. cell segmentation, and are simply a measure of pixels that are positive for a marker over a given area. This approach was used when comparing IF and IHC stains, since the same cell segmentation algorithms cannot be applied to both techniques. Pixel-by-pixel data was extracted and analyzed using R package mIFTO (compiled and developed for AstroPath and available at https://github.com/AstropathJHU/mIFTO). Positive pixels (signal) and negative pixels (noise) were assigned using thresholds determined using inForm's Colocalization Module. Tumor cell expression was studied using a machine learning algorithm to classify pixels into tissue categories. This was required for accurate tumor quantification due to the variation in tumor cell size and the use of a dual marker (Sox10/S100) cocktail, precluding thresholding on a single marker's intensity.

To compare monoplex IF and chromogenic staining a pixel-based approach was used. For the Sox 10/S100 stain, the machine learning algorithm was also used. For all other markers, machine learning was not used for this specific comparison. The number of positive pixels from chromogenic staining was considered baseline, and the percent deviation in positive pixels when using an IF stain was calculated.

Positive signal from monoplex and multiplex IF staining was compared using pixel-based and cell-based approaches. Potential changes in marker intensities between the multiplex and monoplex IF were assessed by comparing the usable dynamic range of each epitope, defined as the difference in mean cell fluorescence intensities of the 95th and 5th percentile per HPF.

### Statistical Analyses

For staining comparisons between corresponding fields acquired from sequential slides paired student t-tests were performed and data were reported as mean±SEM.

### Example 4—Image Acquisition, Phenotyping, and Batch-to-Batch Normalization

### Image Acquisition

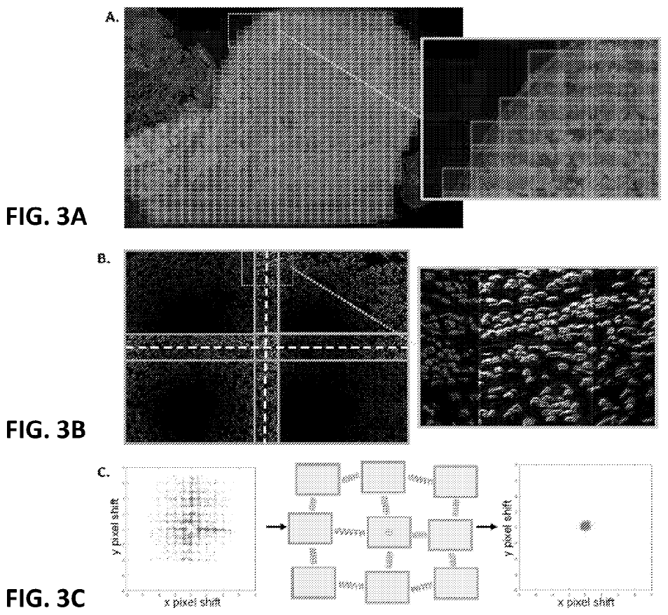

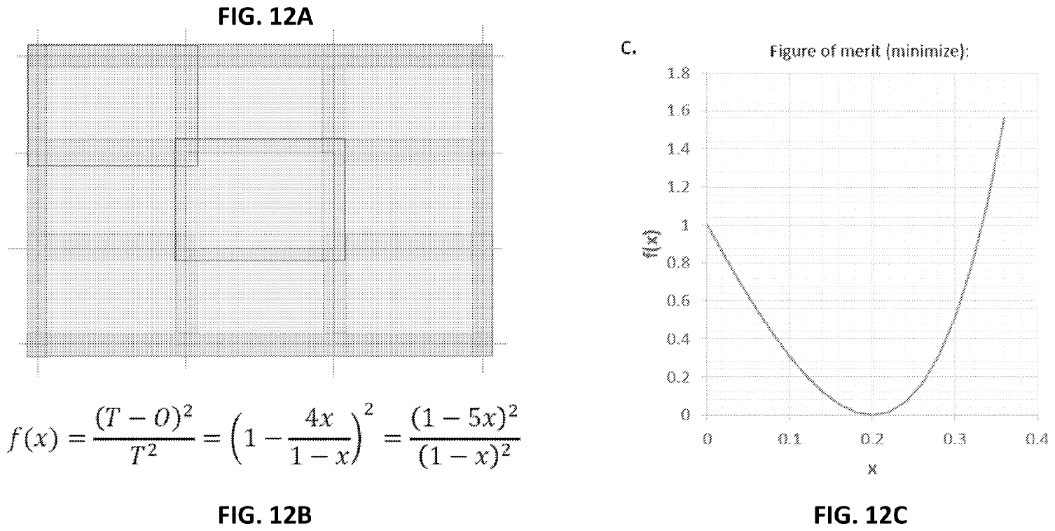

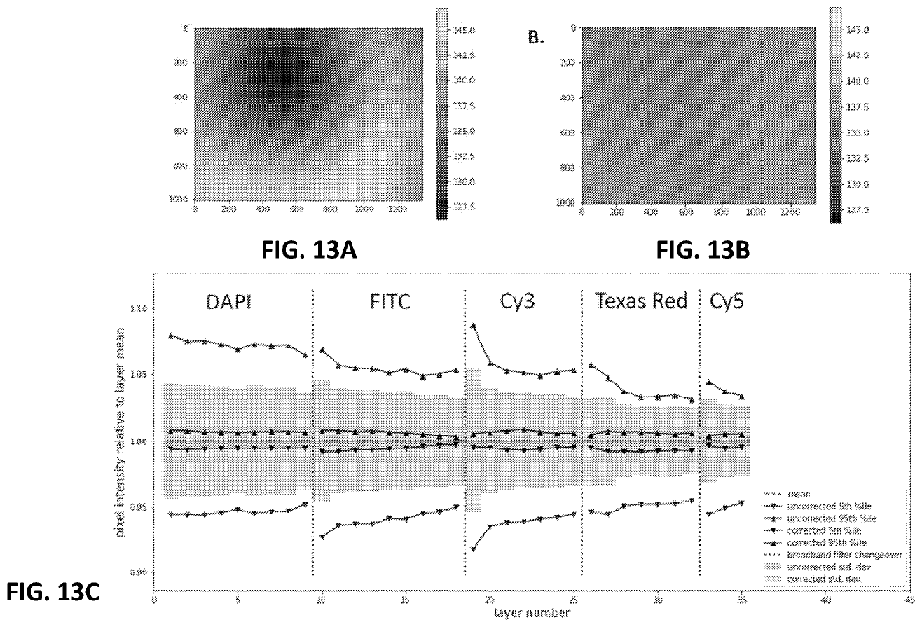

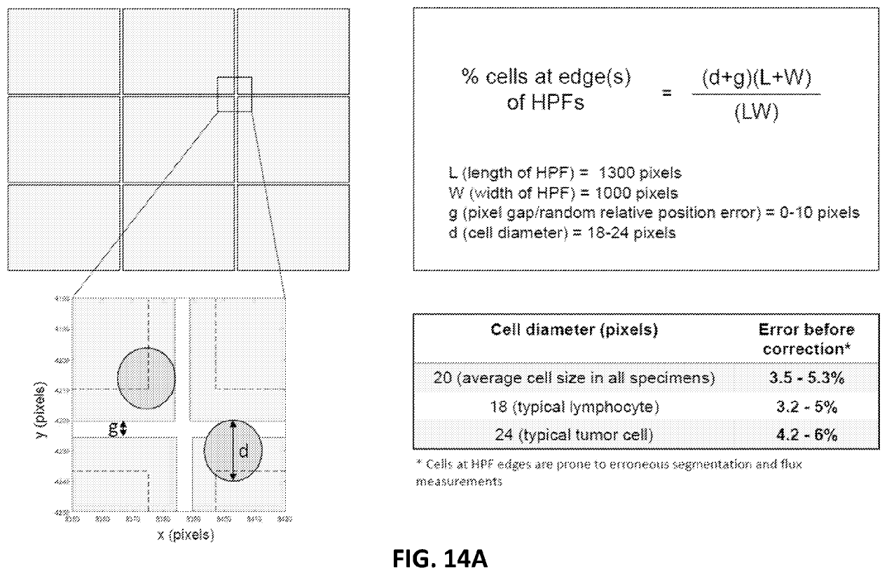

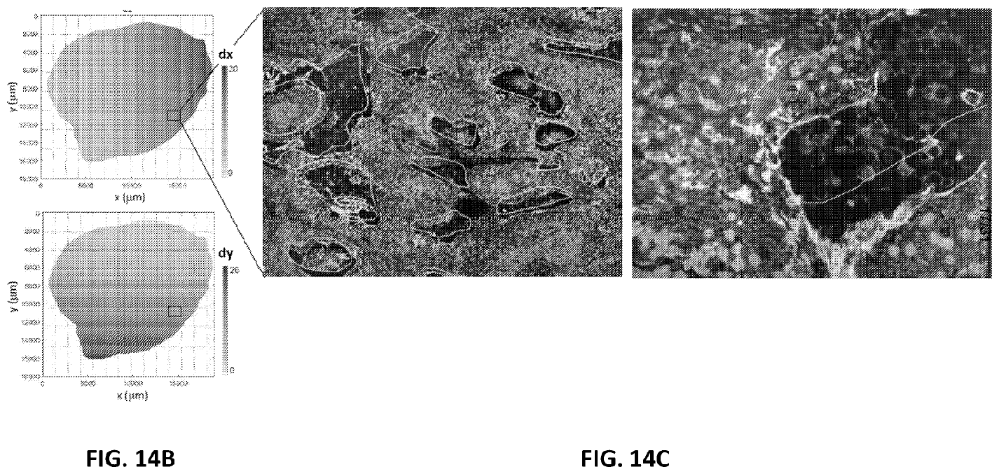

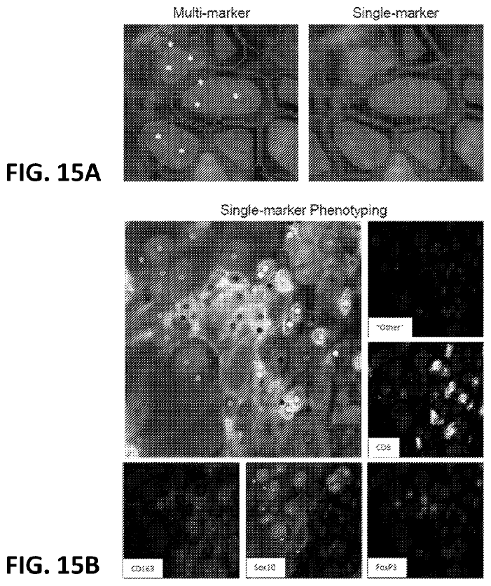

The entire slide was acquired by tiling HPFs with 20% overlap, FIG. 3A and FIGS. 12A-12C. The mid-point of the overlaps was used to determine the boundaries of modified HPFs, FIG. 3B. A flat-field correction for each of the 35 layers was derived from the average of 11,000 HPFs, smoothed by a Gaussian to reduce effects of outliers, FIG. 3B and FIGS. 13A-13C. Mathematical corrections were also applied for ‘pin cushion effects’ resulting from lens distortion for each HPF, FIG. 3B. Fields were then stitched together using a spring-based model that eliminates “jitter” from the microscope stage movement, FIG. 3C and FIGS. 14A-14C.

### Tissue Annotation

The tumor-stroma boundary was manually annotated using HALO (Indica Labs, NM) image analysis software. Areas of necrosis, tissue folds and other artifacts were excluded from analysis.

### Single-Marker Phenotyping and Associated Quality Assurance Quality Control (OA QC)

The inForm software typically assigns phenotypes to individual cell lineages, e.g. CD8 vs. CD163, simultaneously (i.e. ‘Multi-marker’ phenotyping). ‘Single-marker’ phenotyping was also performed, whereby cells were assigned positive or negative status for each marker individually. Cell centers were then used to merge the six individual datasets into a single Cartesian coordinate system.

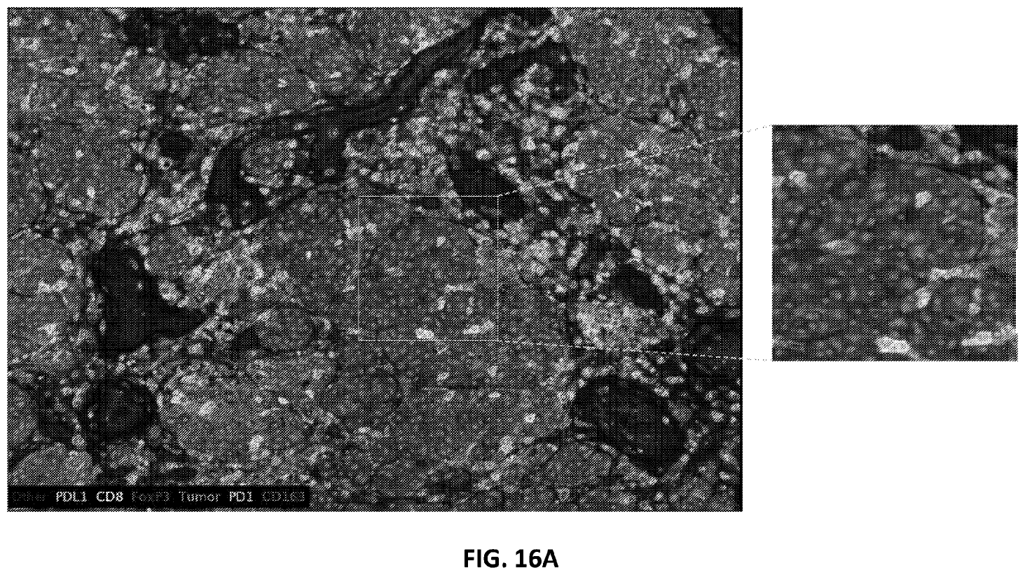

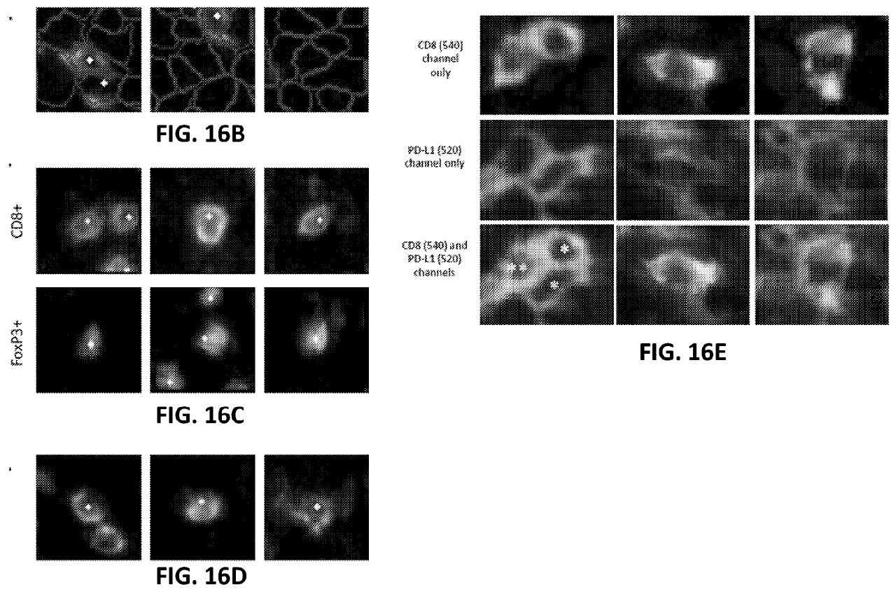

The quality of the final phenotyping was verified by a board-certified pathologist who visually inspected an average of 25,000 phenotyped cells per specimen using a custom viewer, FIG. 16A. Specifically, the 20 highest density CD8 HPFs containing at least 60 tumor cells, 50% tissue coverage, and 400 cells total were selected for each specimen for visual QA/QC inspection of phenotyping algorithm performance. A second custom viewer facilitated inspection of up to randomly selected positive and negative cells for each marker from the same HPFs, FIG. 16B-16D. A minimum of 2000 cells displaying each marker was visually inspected using this second viewer for each specimen. The custom QA/QC code for both viewers can be found at https://github.com/AstropathJHU/MaSS.

### Normalization of Batch-to-Batch Variation

A tissue microarray (TMA) that included 3 normal spleen and 3 tonsils was run with each multiplex staining batch. The staining intensities for PD-1 and PD-L1 in the control tissues were used for batch-to-batch normalization.

### Computing Hardware and Software Configurations

Images were acquired using a local desktop computer associated with the Vectra that was upgraded to contain two 2 TB M.2 NVMe SSDs allocated as a single drive, for maximum storage and transfer efficiency. The multispectral image tiles were then transferred from the local computer to a cluster of 4 servers, dedicated to processing of the Vectra data. Two of the servers were configured for computational performance outfitted with nine 2 TB NVME SSDs, 128 GB of RAM and 24 physical cores. The other two servers were configured for storage, containing six 6×6 TB HDDs configured as RAID5 arrays. This allowed a total net usable HDD capacity of 313.3 TB. This study consumed 32.27 TB of this storage capacity at peak.

One computational server was specifically dedicated to image correction and segmentation, running multiple virtual machines, each with its own inForm instances. The interactive aspects of inForm were overridden using an automation tool, so they could be executed as batch processes. The other computational machine was dedicated to house the database. One of the storage machines contained the compressed backups of the raw data. Each image was compressed individually, to increase accessibility, using settings in the 7-Zip software for optimal speed and compression size for the image files. The final storage server housed the data during processing.

The intermediate data products are reproducible, and can be discarded throughout or after processing; leaving minimum storage requirements for this project around 15 TB without compression. While the configuration expedited image processing and analysis by 12-15 fold using a lot of parallelism, it is important to note that the general workflow described herein could be executed using a single computer outfitted with a single inForm license.

### Example 5—Density Assessments of Cell Types by Distance to the Tumor-Stromal Border

The density of specific cell types expressing PD-1 or PD-L1 was determined relative to the distance from the tumor-stromal border. PD-1 levels (negative, low, medium, and high) were determined by dividing the positive signal for PD-1 into tertiles. To enable comparisons between cell types with varying levels of abundance, a probabilistic density was calculated by dividing the cell density in each distance bin by the total surface density of that cell category.

### Example 6—Density Assessments for Specific Cell Populations and Association with Response to Anti PD-1

The density of specific cell types, including assessments of PD-1 and PD-L1 expression levels (negative, low, mid, high) were determined for each specimen and tested for an association with response to therapy. The assessment of PD-1 and PD-L1 expression levels as low, mid, or high were determined by grouping all the positive cells for either marker from all cases and dividing the dynamic range of positive signal of each into tertiles (FIGS. 18A-18B). The densities of cells displaying the different PD-1/PD-L1 expression levels for each cell type were then compared between responders and non-responders using a one-sided Wilcoxon rank-sum test. The rank sum values were converted into AUC values.

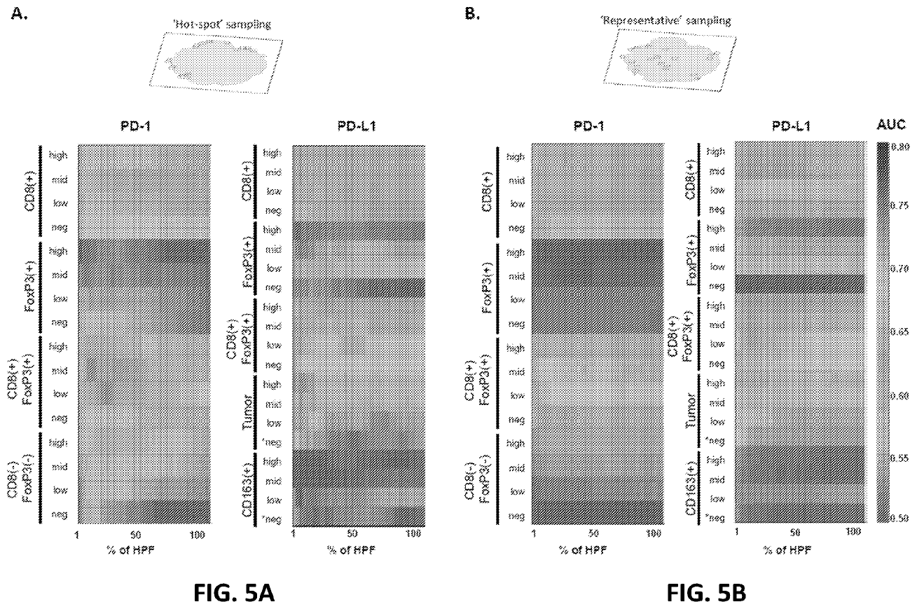

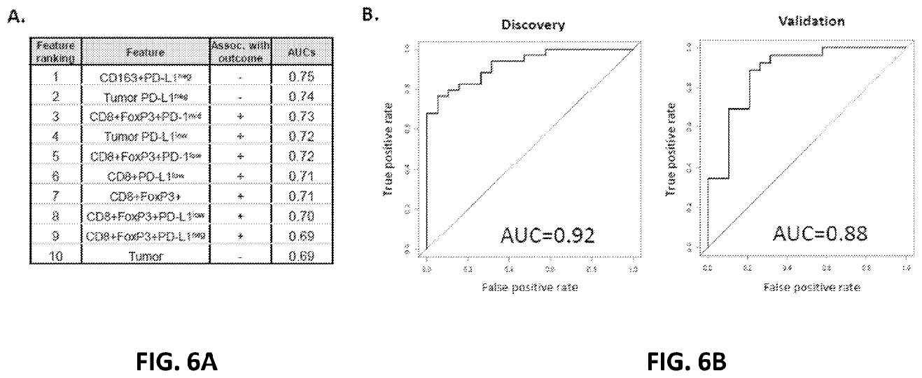

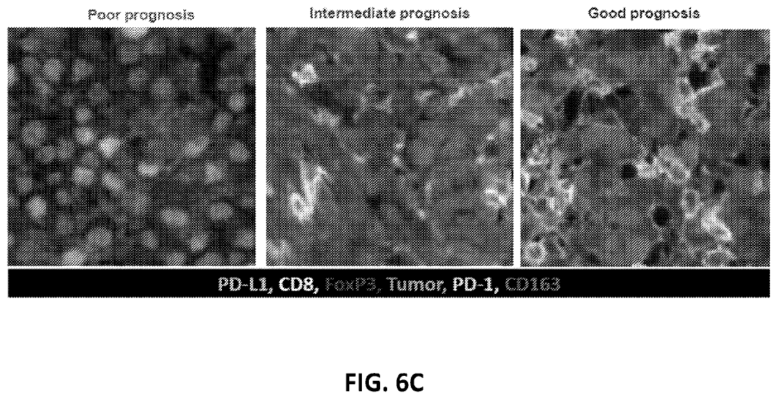

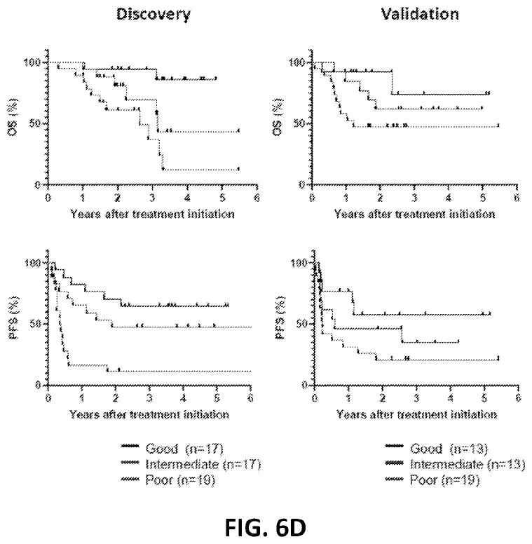

To determine the impact of HPF sampling on the resultant AUC, an increasing proportion of the tumor microenvironment was assessed in an iterative manner. Field sampling was performed in one of two ways. 1) CD8+ cell densities were determined for each HPF and then fields were ranked and included by order of decreasing CD8+ cell densities in the ‘hot-spot’ analysis. 2) Fields were ranked randomly and selected at increasing proportions (FIGS. 5A-5B). To avoid bias, 100 randomized orderings were generated and an average AUC was reported at each proportion step. They were selected randomly for ‘representative’ analysis. Reported p-values are corrected for multiple comparisons using a Benjamini-Hochberg correction.

Each feature that showed an association with response by univariate analysis (corrected p-value <0.05) at 30% hot spot HPFs sampling and for the whole TME (100% sampling) was combined into a multivariate model. Specifically, a binary logistic regression model was applied to assess the combinatorial ROC curves and the corresponding AUCs were calculated evaluate the prognostic accuracy of combination of the top 10 features in the Discovery cohort for predicting objective response. These same 10 features were then tested in an independent validation cohort. A combined model was also developed using these features for predicting long-term survival by Kaplan Meier analysis. In this combinatorial model, patients whose samples contained high densities (top 20%) for any one of the features negatively associated with outcome were grouped together first, irrespective of other expressed factors. Next, the remaining patients were divided between those containing high densities (top 15%) for any one of the features positively associated with outcome.

### Example 7—mIF Assay for PD-1 Expression by Lymphocyte Subsets

A six-plex mIF assay for PD-1, CD8, CD4, CD20, FoxP3, and tumor (Sox10/S100) was developed and validated on an automated platform (Leica Bond Rx). The staining order and conditions for staining are provided in Table 7. This was used to assess the proportion of PD-1 expression contributed by individual lymphocyte subsets to the melanoma TME.

TABLE 7

| | | Primary Antibody | | | | | Secondary Antibody | | | TSA Fluorophone | |
|---|---|---|---|---|---|---|---|---|---|---|---|
| | | | | | Final | Incubation | | | Dilution | | TSA |
| Position | Market | Species | Clone | Source | (μg/mL) | times (min) | Amplification | Source | (in PBS) | Opal | dilution |
| 1 | FoxP3 | Mouse | 236A/E7 | abcam | 5.67 | 30 | Mouse | Leica | 1: [symbol] | 570 | 1: [symbol] |
| | | | | inc. | | | Powervision | Biosystems | | | |
| 2 | CD8 | Mouse | 4811 | Leica | 0.2 [symbol] | 60 | Opal | Akoya | None | 620 | 1:300 |
| | | | | Biosystems | | | Polymer | Biosciences | | | |
| 3 | CD20 | Mouse | L25 | Leica | 0.24 | 60 | Opal | Akoya | None | 520 | 1:300 |
| | | | | Biosystems | | | Polymer | Biosciences | | | |
| 4 | PD-1 | Rabbit | EPF4877 | abcam | 0.96 | 60 | Rabbit | Leica | 1:5 | 650 | 1:250 |
| | | | | inc. | | | Powervision | Biosystems | | | |
| 5 | CD4 | Rabbit | EP204 | Millipore | 0.18 | 120 | Rabbit | Leica | 1:5 | 540 | 1:200 |
| | | | | [symbol] | | | Powervision | Biosystems | | | |
| [symbol] | Sox10 | Mouse | BC34 | BioCare | 0.06 | 60 | Opal | Akoya | None | 690 | 1: [symbol] 50 |
| | | | | Medical | | | Polymer | Biosciences | | | |
| | S100 | Mouse | 4C4.9 | Abnova | 0.06 | | | | | | |
| | | | | Corp | | | | | | | |

[symbol]

indicates data missing or illegible when filed

### Example 8—Multiplex IF Staining of Slides

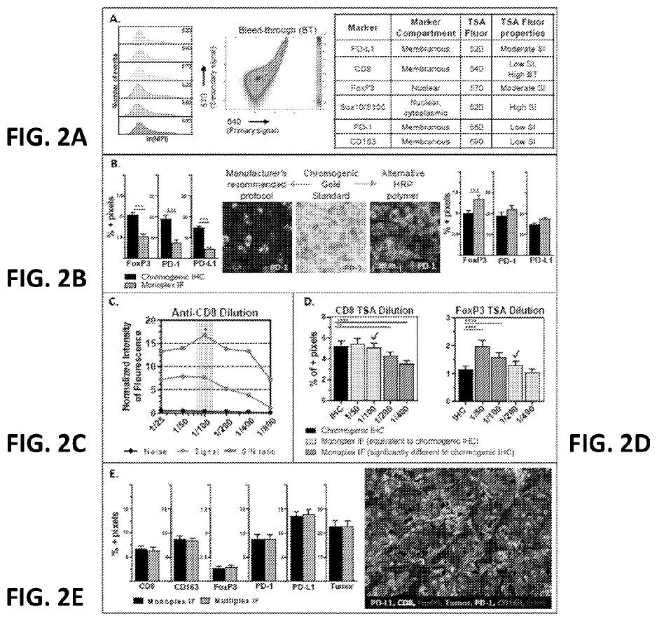

During the staining process, sources of potential error arise when signal is not fully detected or when false positive signal is detected in a given channel due to spillover from a different channel, a.k.a. ‘bleed-through’. The design and optimization of the 6-plex panel therefore involved 1) determination of a staining index (SI) for each fluorophore and pairing of TSA fluorophores with markers based on bleed-through calculation, 2) selection of secondary/amplification reagents, as well as selection of the concentration of 3) primary antibody and 4) fluorophores for maximal sensitivity and specificity. The final step is the combination of all the optimized monoplex protocols into the multiplex assay format such that equivalent staining is achieved for each marker between 6-plex mIF, monoplex IF, and single stain chromogenic IHC (FIGS. 2A-2E and Table 8).

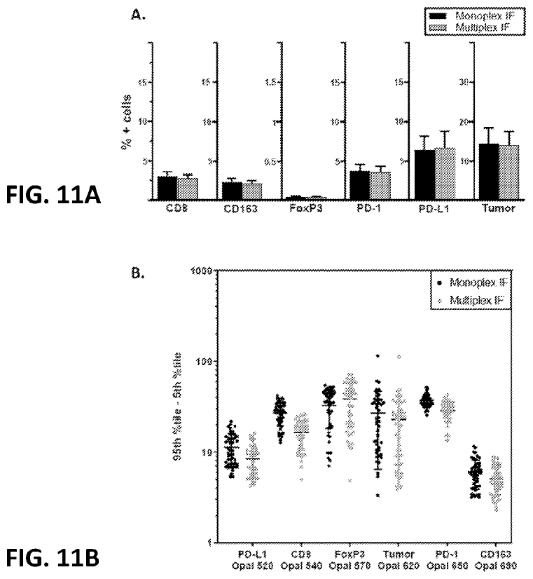

TABLE 8

| Task | Commercial Kit/ | | Example of contribution/ |
|---|---|---|---|
| (in order of | Manufacturer | | improvement beyond |
| operation*) | recommendation | AstroPath recommendation | Commercial platform |
| Pairing marker to | Exposure time 50-250 ms. | Consideration given to (1) | Facilitates balancing of signals |
| fluorophore | orintensity 5 to 30 after | fluorophore staining index, (2) | through pairing stronger flours |
| | primary antibody | target protein expression | with weaker markers and vice |
| | optimization. | intensity, and (3) subcellular | versa. Also, facilitates |
| | | location (nucleus vs. | mitigation of any potential |
| | | membrane). | residual bleed-through at later |
| | | | stages of panel development by |
| | | | capitalizing on differential |
| | | | subcellular localization, FIGS. |
| | | | 9A-9B. |
| Selection of secondary | Secondary antibody | Select markers require | Capture populations with lower |
| antibody | provided with commercial | replacement of commercial kit | levels of marker expression |
| | kit. | secondary antibody with an | (e.g. PD-llow/mid), improving |
| | | alternative to meet ‘gold- | sensitivity by 50%, FIGS. 2A- |
| | | standard’ chromogenic IHC. | 2E and FIGS. 18A-18B. |
| Primary antibody | See “Pairing of marker to | Titration of primary antibody | Improved sensitivity and |
| optimization | fluorophore” above. | to optimize the signal to noise | specificity through optimized |
| | | ratio (SNR), FIGS. 10A-10B. | SNR e.g. Sox10 had a 3 fold |
| | | | higher SNR when using our |
| | | | approach vs. manufacturer |
| | | | recommendation (optimal |
| | | | concentration resulted in |
| | | | intensity counts of up to 100). |
| TSA optimization | Recommended dilution of | Titration of TSA to identify | Improved specificity through |
| | 1:100 (recent update).** | concentration required to | reduced false positive signal |
| | | reduce potential bleed-trough | (bleed-through) from adjacent |
| | | and steric hindrance (with | channel, e.g. reduced 4-fold |
| | | excess TSA) without signal | (12% to 3%) from 570 (FoxP3) |
| | | loss (with insufficient TSA). | to 540 (CD8) channel. The |
| | | | remaining 3% is further |
| | | | reduced during image analysis |
| | | | by capitalizing on differential |
| | | | subcellular localization (see |
| | | | pairing marker to fluorophore |
| | | | above). |
| Validation of final mIF | None provided. | mIF validated against | mIF panel sensitivity and |
| panel against | | chromogenic IHC.*** | specificity is comparable to |
| chromogenic IHC | | | gold-standard, FIGS. 2A-2E |
| | | | and FIGS. 11A-11B. |

First, the propensity of each marker for bleed-through was determined, FIG. 9A-9B. The SI and bleed-through information was then used to pair TSA fluorophores with markers, FIG. 2A. For example, a fluorophore with high SI was paired with a marker with lower intensity expression, e.g. TSA fluorophore 520 and PD-L1. Fluorophore pairs ‘at risk’ for bleed-through were assigned to markers found in different cellular compartments, allowing any potential bleed-through to be removed during image analysis, e.g. CD8, a membrane stain, was paired with TSA fluorophore 540, while FoxP3, a nuclear stain, was paired with TSA fluorophore 570.

The critical next step is evaluation of the secondary antibody/amplification reagent. For example, when using a ‘less powerful’ secondary antibody/HRP polymer system, only 50% of PD-1 expressing cells were identified compared to chromogenic IHC, FIG. 2B. PD-L1 and FoxP3 also showed lower levels of expression, while all other markers showed comparable staining between monoplex IF and chromogenic IHC. To address the relative loss of detection of PD-1, PD-L1, and FoxP3, different components of the assay were modified, including the primary and secondary antibody reagents, incubation times, and different amplification methods. A new secondary antibody (PowerVision Poly-HRP, 1:1 dilution, Leica Biosystems) improved the assay sensitivity for these markers, FIG. 2B, and thus was adopted for PD-1, PD-L1, and FoxP3 in the panel. Importantly, it was found that it was key to select the secondary antibody for each marker prior to primary antibody or TSA dilution optimization. The primary antibody concentration is determined next, FIG. 2C and FIGS. 10A-10B, followed by selection of the TSA concentration for each fluorophore, FIG. 2D. These latter two steps serve to optimize the signal to noise ratio and to prevent signal bleed-through or blocking, respectively.

The final step in assay validation is to combine all of the optimized monoplex protocols into the multiplex assay format. When following the approach described herein, equivalent staining is achieved for each marker between 6-plex mIF, monoplex IF, and single stain chromogenic IHC, FIG. 2E and FIG. 11A. Of note, while the total cell counts in multiplex format matched those in monoplex, the dynamic range (as representative of the intensity spread between the 95th and 5th percentile cell expressing a given marker) of the immunofluorescence signal was lower in the multiplex vs. monoplex format, FIG. 11B.

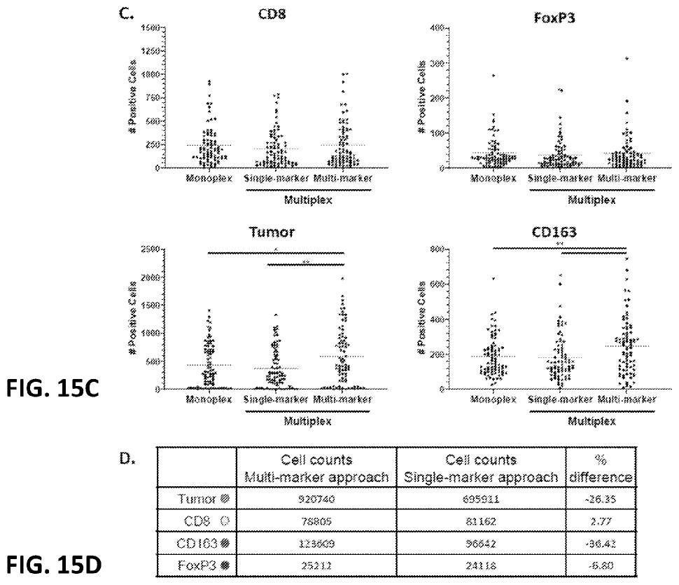

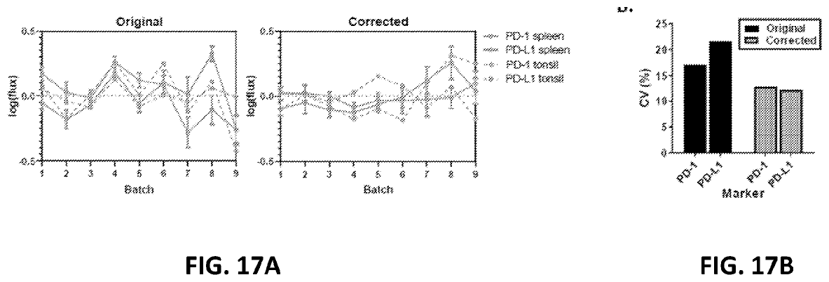

TABLE 9

| | | Commercial (software) or | | Contributions/ |
|---|---|---|---|---|
| | | custom | | improvements beyond |
| | Task | (GitHub code name) | Description | commercial platform |
| Image | Modified | Custom | Change settings in the | Facilitates image |
| acquisition | image | (Phenochart.config*) | Phenochart software ROI | corrections and whole |
| and | acquisition | | functionality to generate 20% | slide stitching, including |
| processing | protocol | | HPF overlap | accurately mapping the |
| of | | | (qpTIFF output) | 3-6% of cells found at |
| individual | | | | HPF edges, FIGS. 12A- |
| HPFs | | | | 12C, FIGS. 13A-13C, |
| | | | | and FIGS. 14A-14C. |
| | Image | Commercial | Image the slides using the Vectra | N/A |
| | acquisition | (Vectra 3.0 software) | platform | |
| | | | (im3 file output) | |
| | Spectral | Commercial | Deconvolution of spectral | N/A |
| | unmixing of | (inForm) | signatures for the seven detected | |
| | image | | colors (6 markers + DAPI) and | |
| | | | removal of autofluorescence | |
| | | | (component TIFF output) | |
| | Image | Custom | Correct the images for | This step reduces |
| | correction/ | (flatw*) | illumination variation and lens | systematic error in the |
| | processing | | distortion effects | HPFs themselves, e.g. |
| | | | | illumination variation |
| | | | | reduced 9x (11.2% → |
| | | | | 1.2%), FIGS. 13A- |
| | | | | 13C. |
| Lineage | Segmentation | Commercial | Commercial cell segmentation | When run as a single |
| assignment | and | (with modified usage) | and phenotyping routine is run | pass, the cell |
| and | phenotyping | | multiple times (segmentation for | segmentation/ |
| normalized | | | larger vs. smaller cells, and | phenotyping algorithm |
| PD-1/L1 | | | phenotyping once for each | overestimates the |
| expression | | | marker), i.e. a “multipass | number of large cells |
| levels per | | | approach” for each | (tumor cells and |
| individual | | | | macrophages) by 25%, |
| cell | | | | FIGS. 15A-15D. |
| | Merge | Custom | Outputs from the cell | Multipass phenotyping |
| | multipass | (MaSS**) | segmentation/phenotyping | allows for training for |
| | data | | routine for each marker are | each marker |
| | | | merged into a single data set | individually. Individual |
| | | | | markers are then |
| | | | | combined at this stage, |
| | | | | simplifying training |
| | | | | algorithms and |
| | | | | facilitating the |
| | | | | identification of rare cell |
| | | | | phenotypes. |
| | QA/QC | Custom | Shows images to visually inspect | Commercial platform |
| | phenotyping | (Create image QA/QC**) | performance of multipass | cannot be used to |
| | | | phenotyping and merging | visually inspect results |
| | | | algorithms. | of multipass approach. |
| | | | | Also, functionality |
| | | | | showing individually, |
| | | | | randomly selected |
| | | | | positive and negative |
| | | | | cells for each marker is |
| | | | | provided, |
| | | | | FIGS. 16A-16E. |
| | Batch | Custom | Reduce potential batch-to-batch | Average batch-to-batch |
| | Normalization | (calib*) | intensity variation by | variation for PD-1 and |
| | | | normalizing to control tissues | PD-L1 expression |
| | | | | intensity was ~20%, and |
| | | | | this was reduced by half |
| | | | | through normalization, |
| | | | | FIGS. 17A-17B. |
| Image | Image | Custom | Seamless stitching of image tiles | This step corrects the |
| handling | stitching and | (align, shift*) | into a whole slide. Scaling all | combination of different |
| and | mapping to | | inputs (multiple scanners, | errors generated when |
| individual | absolute | | images, and annotations) to an | re-assembling numerous |
| cell | coordinate | | absolute Cartesian coordinate | HPFs into a single |
| mapping in | system | | system*** | image. (~5% loss of cells |
| whole-slide | | | | around perimeter of |
| format | | | | HPFs, and 40 □m |
| | | | | cumulative shift with |
| | | | | regard to relative cell |
| | | | | position from the left to |
| | | | | right edge of a whole |
| | | | | slide). |
| | Image | Commercial | Pathologist manual annotation of | N/A |
| | annotation | (Halo) | tumor-stromal boundary and | |
| | | | removal of tissue artifacts (tears, | |
| | | | folds, etc) (qpTIFF output) | |
| | Image | Custom | Pathologist manual annotation of | Annotations are stored in |
| | annotation | (prepdb*) | tumor-stromal boundary, etc is | the database, lending |
| | overlay | | applied to whole slide, stitched | ease to spatial statistics |
| | | | image | and visualizations. This |
| | | | | makes data consistency |
| | | | | easier and is of particular |
| | | | | interest for anticipated |
| | | | | tumor-immune Atlas |
| | | | | generation and use*** |

TABLE 10

| Feature | AUC | Uncorrected p value | Benjamini-Hochberg corrected p value | Association with response |
|---|---|---|---|---|
| CD163_PDL1_neg | 0.751 | 0.001 | 0.036 | − |
| Tumor_PDL1_neg | 0.743 | 0.002 | 0.036 | − |
| CD8FoxP3_PD1_mid | 0.725 | 0.004 | 0.036 | + |
| Tumor_PDL1_low | 0.721 | 0.004 | 0.036 | + |
| CD8FoxP3_PD1_low | 0.717 | 0.005 | 0.036 | + |
| CD8_PDL1_low | 0.711 | 0.006 | 0.036 | + |
| CD8FoxP3 | 0.710 | 0.006 | 0.036 | + |
| CD8FoxP3_PDL1_low | 0.695 | 0.009 | 0.045 | + |
| CD8FoxP3_PDL1_neg | 0.693 | 0.011 | 0.045 | + |
| Tumor | 0.692 | 0.011 | 0.045 | − |
| CD8_PD1_neg | 0.681 | 0.015 | 0.057 | + |
| CD163 | 0.675 | 0.018 | 0.063 | − |
| CD8FoxP3_PD1_neg | 0.672 | 0.020 | 0.064 | + |
| CD8 | 0.661 | 0.027 | 0.075 | + |
| CD8FoxP3_PDL1_mid | 0.661 | 0.027 | 0.075 | + |
| CD8_PD1_low | 0.656 | 0.031 | 0.077 | + |
| CD8_PDL1_mid | 0.653 | 0.034 | 0.077 | + |
| CD8FoxP3_PD1_high | 0.653 | 0.034 | 0.077 | + |
| CD8_PDL1_neg | 0.644 | 0.043 | 0.093 | + |
| CD8_PD1_mid | 0.635 | 0.054 | 0.106 | + |
| CD8FoxP3_PDL1_high | 0.634 | 0.054 | 0.106 | + |
| CD8_PDL1_high | 0.602 | 0.112 | 0.209 | + |
| FoxP3_PDL1_low | 0.596 | 0.127 | 0.226 | + |
| Other_PD1_high | 0.593 | 0.135 | 0.230 | + |
| Other_PD1_mid | 0.573 | 0.194 | 0.314 | + |
| Tumor_PDL1_mid | 0.571 | 0.199 | 0.314 | + |
| CD8_PD1_high | 0.567 | 0.215 | 0.315 | + |
| Other_PD1_low | 0.567 | 0.215 | 0.315 | + |
| Tumor_PDL1_high | 0.563 | 0.226 | 0.320 | + |
| FoxP3_PD1_neg | 0.560 | 0.238 | 0.325 | + |
| FoxP3_PDL1_mid | 0.554 | 0.261 | 0.342 | + |
| FoxP3 | 0.553 | 0.267 | 0.342 | + |
| FoxP3_PD1_low | 0.548 | 0.286 | 0.355 | + |
| FoxP3_PD1_high | 0.533 | 0.352 | 0.405 | + |
| CD163_PDL1_low | 0.533 | 0.352 | 0.405 | + |
| FoxP3_PD1_mid | 0.529 | 0.366 | 0.405 | + |
| Other_PD1_neg | 0.529 | 0.366 | 0.405 | + |
| FoxP3_PDL1_neg | 0.525 | 0.387 | 0.407 | + |
| FoxP3_PDL1_high | 0.525 | 0.387 | 0.407 | + |
| CD163_PDL1_high | 0.508 | 0.467 | 0.478 | + |
| CD163_PDL1_mid | 0.503 | 0.489 | 0.489 | + |

TABLE 11

| Feature | AUC | Uncorrected p value | Benjamini-Hochberg corrected p value | Association with response |
|---|---|---|---|---|
| CD163_PDL1_neg | 0.759 | 0.001 | 0.041 | − |
| Tumor_PDL1_neg | 0.726 | 0.003 | 0.043 | − |
| Tumor_PDL1_low | 0.721 | 0.004 | 0.043 | + |
| CD163 | 0.715 | 0.005 | 0.043 | − |
| CD8FoxP3_PD1_mid | 0.709 | 0.006 | 0.043 | + |
| CD8_PDL1_low | 0.709 | 0.006 | 0.043 | + |
| CD8FoxP3 | 0.699 | 0.009 | 0.049 | + |
| CD8FoxP3_PD1_low | 0.697 | 0.009 | 0.049 | + |
| CD8FoxP3_PDL1_low | 0.688 | 0.012 | 0.054 | + |
| Tumor | 0.681 | 0.015 | 0.061 | − |
| CD8FoxP3_PDL1_neg | 0.679 | 0.016 | 0.061 | + |
| CD8_PD1_neg | 0.663 | 0.026 | 0.090 | + |
| CD8FoxP3_PDL1_mid | 0.647 | 0.039 | 0.108 | + |
| CD8FoxP3_PD1_high | 0.646 | 0.041 | 0.108 | + |
| CD8_PDL1_mid | 0.645 | 0.041 | 0.108 | + |
| CD8FoxP3_PD1_neg | 0.644 | 0.043 | 0.108 | + |
| CD8 | 0.642 | 0.045 | 0.108 | + |
| CD8FoxP3_PDL1_high | 0.634 | 0.054 | 0.122 | + |
| CD8_PD1_low | 0.630 | 0.061 | 0.131 | + |
| CD8_PDL1_neg | 0.627 | 0.065 | 0.134 | + |
| CD8_PD1_mid | 0.616 | 0.084 | 0.163 | + |
| CD8_PDL1_high | 0.587 | 0.152 | 0.283 | + |
| Tumor_PDL1_mid | 0.582 | 0.165 | 0.294 | + |
| Tumor_PDL1_high | 0.557 | 0.249 | 0.418 | + |
| FoxP3_PDL1_low | 0.556 | 0.255 | 0.418 | + |
| CD8_PD1_high | 0.551 | 0.273 | 0.424 | + |
| Other_PD1_high | 0.550 | 0.280 | 0.424 | + |
| Other_PD1_mid | 0.543 | 0.305 | 0.435 | + |
| FoxP3_PDL1_mid | 0.542 | 0.312 | 0.435 | + |
| CD163_PDL1_low | 0.540 | 0.318 | 0.435 | + |
| Other_PD1_low | 0.529 | 0.366 | 0.484 | + |
| FoxP3_PD1_neg | 0.517 | 0.423 | 0.520 | + |
| FoxP3_PD1_low | 0.517 | 0.423 | 0.520 | + |
| FoxP3_PDL1_high | 0.512 | 0.445 | 0.520 | + |
| FoxP3 | 0.511 | 0.452 | 0.520 | + |
| FoxP3_PD1_mid | 0.509 | 0.459 | 0.520 | + |
| CD163_PDL1_high | 0.506 | 0.474 | 0.520 | + |
| CD163_PDL1_mid | 0.505 | 0.482 | 0.520 | + |
| FoxP3_PDL1_neg | 0.491 | 0.548 | 0.576 | + |
| FoxP3_PD1_high | 0.485 | 0.577 | 0.592 | + |
| Other_PD1_neg | 0.472 | 0.634 | 0.634 | + |

### Example 9—CD8+FoxP3+PD-1+ Cells Strongly Associate with Objective Response for Patients with Advanced Non-Small Cell Lung Cancer Treated with Anti-PD-1-Based Therapy

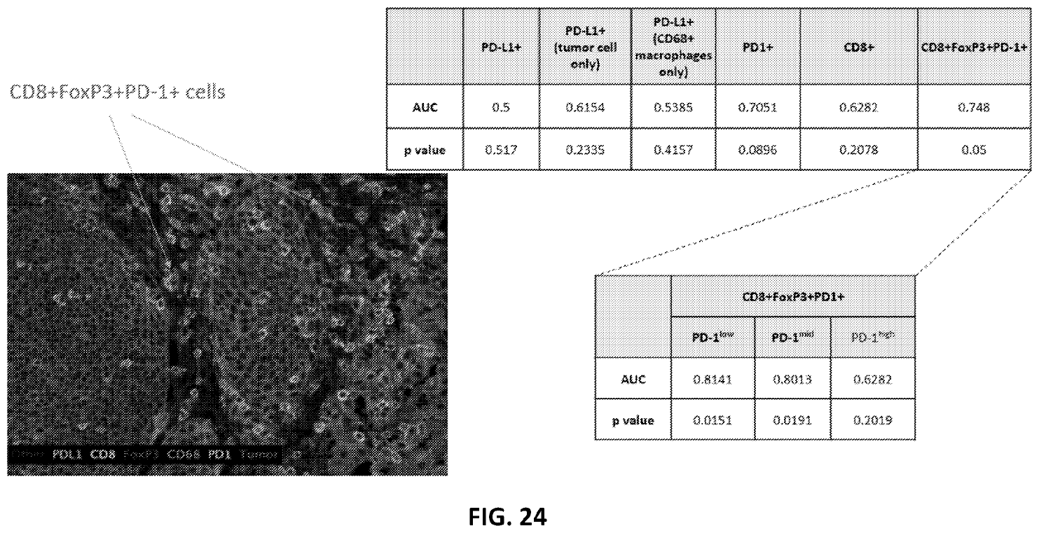

Pre-treatment lung cancer specimens from n-20 patients with advanced disease were stained with a 6-plex mIF assay, and the entire specimen was imaged using a tiled mosaic of high power fields (HPFs). In this example, the 20 HPFs with the highest CD8 cell densities were selected for continued analysis. The density of PD-1 and PD-L1 expressing cell populations within the HPF tiles were assessed for their predictive value for objective response (determined by the area under the curve (AUC) of a receiver operator characteristic curve). Of those populations studied, the population showing the closest association with a positive response to therapy was the CD8+FoxP3+ cells expressing PD-1. When this population was further resolved into those expressing PD-1 at different tertile expression levels (low, mid, high), the PD-1low and PD-1mid expressing cells were most closely associated with response (FIG. 24). The image corrections facilitated by the approach described herein allow for such robust and reproducible assessments of marker expression intensity in situ. This finding is of note, as it extends the findings observed in melanoma into non-small cell lung cancer. Similar findings were observed in advanced Merkel cell carcinoma, supporting the pan-tumor biomarker significance of this cell population and imaging and analysis approach.

### Example 10—AstroPath Imaging Approach Applied to Pre-Treatment Specimens from Patients with Non-Small Cell Lung Cancer Receiving Anti-PD-1

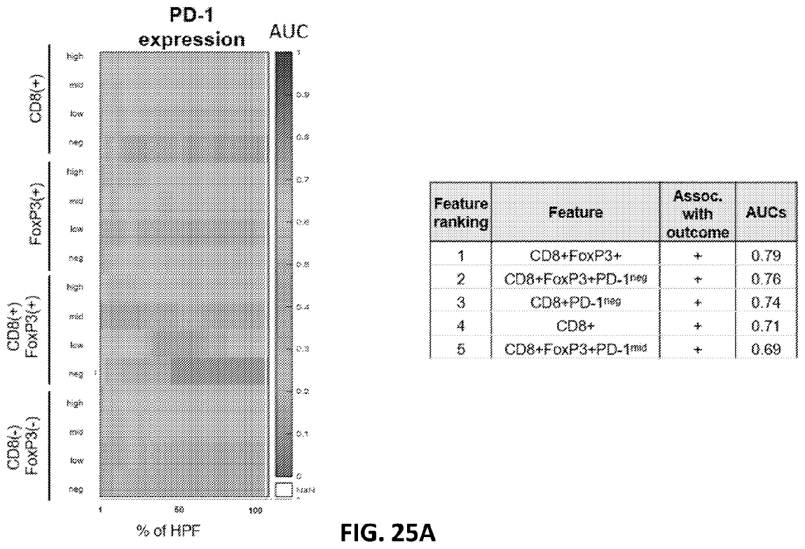

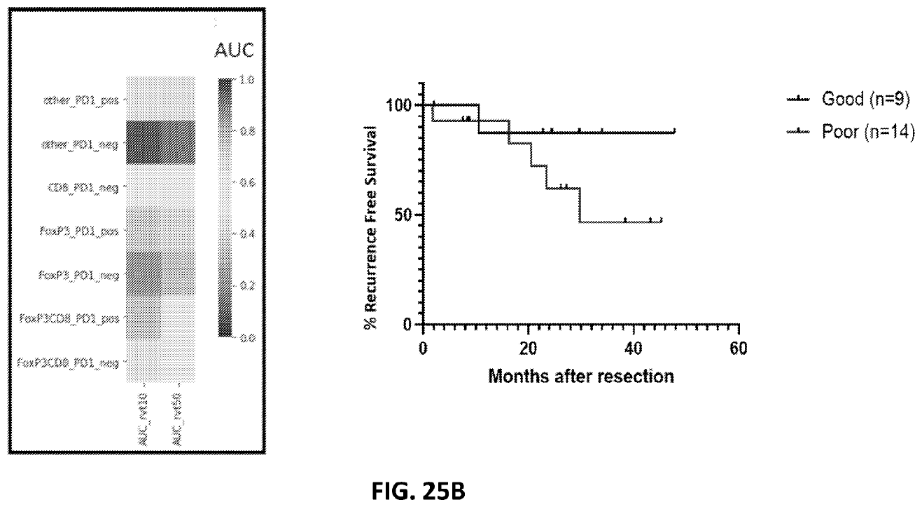

The median pre-treatment biopsy size in this cohort was 3 mm² (average 15 mm²). An assessment of HPF sampling showed that the highest AUCs were achieved when 100% of the pre-treatment HPFs were sampled. In this analysis, the density of all CD8+FoxP3+ cells had the highest predictive value for a positive response for an individual feature identified on the mIF assay (FIG. 25A). A second analysis was performed to determine pre-treatment features that predicted the degree of pathologic response to therapy (FIG. 25B). The heat map showed some potential cell populations identified by this method and their association with pathologic response values of 10% residual viable tumor (rvt10, which is an endpoint of numerous Phase II/III clinical trials) and 50% residual viable tumor (rvt50) (FIG. 25B; Left). The features identified using this approach was used to predict survival outcomes for these patients (Kaplan-Meier analysis) (FIG. 25B; Right).

What is claimed is:

1. A method of predicting a subject's response to immunotherapy, the method comprising:
(a) staining a biological sample disposed on a substrate;
(b) imaging the biological sample, wherein one or more image(s) of a high-power field (HPF) is generated;
(c) detecting multiple biomarkers in the biological sample; and
(d) analyzing the one or more image(s), thereby predicting the subject's response to immunotherapy.

2. A method of stratifying a subject and placing the subject in a therapy category, the method comprising:
(a) staining a biological sample disposed on a substrate;
(b) imaging the biological sample, wherein one or more image(s) of a high-power field (HPF) is generated;
(c) detecting multiple biomarkers in the biological sample; and
(d) analyzing the one or more image(s), thereby stratifying the subject and placing the subject in a therapy category.

3. The method of claim 1 or 2, wherein the multiple biomarkers comprise PD-1, PD-L1, CD8, FoxP3, CD163, a tumor cell marker, or any combination thereof.

4. The method of claim 3, wherein the tumor cell marker comprises Sox10, S100, or both.

5. The method of any of one of claims 1-4, wherein the staining comprises an immunofluorescence stain.

6. The method of any one of claims 1-5, wherein the staining comprises an immunohistochemistry stain.

7. The method of any one of claims 1-6, wherein the biological sample is stained with an antibody.

8. The method of claim 7, wherein the antibody is a monoclonal antibody.

9. The method of claim 7, wherein the antibody is a polyclonal antibody.

10. The method of any one of claims 1-9, wherein the biological sample is stained with one or more antibodies.

11. The method of claim 10, wherein the biological sample is stained with six antibodies.

12. The method of claim 10, wherein the biological sample is stained with four antibodies.

13. The method of any one of claims 1-7, wherein the biological sample is stained with a second antibody which detects the antibody.

14. The method of claim 13, wherein the second antibody is conjugated to a label.

15. The method of claim 14, wherein the label is a detectable label.

16. The method of claim 15, wherein the label is a fluorophore.

17. The method of any one of claims 1-16, wherein the imaging step (c) comprises performing immunofluorescence microscopy on the biological sample.

18. The method of any one of claims 1-17, wherein the analyzing step (d) comprises:
(i) image acquisition and processing;
(ii) cell segmentation and phenotyping; and
(iii) image normalization.

19. The method of claim 18, wherein the step of image acquisition comprises compiling the one or more images to acquire an image of the whole biological sample within the substrate.

20. The method of claim 19, wherein the compiling comprises aligning the one or more images with an overlap.

21. The method of claim 18, wherein the step of cell segmentation and phenotyping comprises identifying a cell type in the biological sample.

22. The method of claim 21, wherein the step of phenotyping comprises detecting expression of at least one of the biomarkers in the cell type.

23. The method of claim 22, wherein the expression of the at least one biomarker is designated as low, medium, or high.

24. The method of claim 21, wherein the cell type comprises a CD163+macrophage, a CD8+ T cell, a Treg cell (CD8negFoxP3+), a tumor cell, a CD8+FoxP3+ cell, or any combinations thereof.

25. The method of claim 21, wherein the cell type of CD8+FoxP3+PD-1 low/mid is identified as an indicator that the subject will respond to the immunotherapy.

26. The method of claim 21, wherein the cell type of CD163+PD-L1 neg is identified as an indicator that the subject will not respond to the immunotherapy to the same extent as a reference subject that is identified as not having a cell type of CD163+PD-L1 neg.

27. The method of claim 21, wherein the step of cell segmentation and phenotyping further comprises determining a density of the cell type in the biological sample.

28. The method of claim 27, wherein the density of the cell type in the biological sample is determined by analyzing a distance between a cell and another cell.

29. The method of claim 27, wherein the density of the cell type in the biological sample is determined by analyzing a distance between a cell and a tumor-stromal boundary.

30. The method of claim 27, wherein a high density of CD8+FoxP3+ cells is identified as an indicator that the subject will respond to the immunotherapy.

31. The method of claim 18, wherein the step of image normalization comprises calibrating a fluorescence intensity of at least one of the biomarkers in the one or more images against a tissue micro array.

32. The method of any one of claims 1-31, wherein the analyzing step (c) further comprises identifying the at least one biomarker in the biological sample from a subject having a disease, and wherein the identification of the at least one biomarker is used to predict the subject's response to immunotherapy and/or stratify the subject and place the subject in a therapy category.

33. The method of claim 32, wherein the disease is a cancer.

34. The method of claim 33, wherein the cancer is a metastatic solid tumor.

35. The method of claim 33, wherein the cancer is a melanoma.

36. The method of claim 33, wherein the cancer is a non-small cell lung cancer.

37. The method of claim 33, wherein the cancer is selected from a bladder cancer, breast cancer, cervical cancer, colon cancer, endometrial cancer, esophageal cancer, fallopian tube cancer, gall bladder cancer, gastrointestinal cancer, head and neck cancer, hematological cancer, Hodgkin lymphoma, laryngeal cancer, liver cancer, lung cancer, lymphoma, melanoma, mesothelioma, ovarian cancer, primary peritoneal cancer, salivary gland cancer, sarcoma, stomach cancer, thyroid cancer, pancreatic cancer, renal cell carcinoma, glioblastoma and prostate cancer.

38. The method of claim 32, wherein the immunotherapy comprises administration of an immune checkpoint inhibitor.

39. The method of claim 32, wherein the therapy category comprises radiation therapy, chemotherapy, immunotherapy, hormone therapy, antibody therapy, or any combination thereof.

40. The method of any one of claims 1-39, wherein the substrate is a slide.

41. The method of any one of claims 1-40, wherein the biological sample comprises a tissue, a tissue section, an organ, an organism, an organoid, or a cell culture sample.

42. The method of claim 41, wherein the tissue is a formalin-fixed paraffin-embedded (FFPE) tissue.

43. The method of any one of claims 1-42, wherein the biological sample is fixed prior to step (a).

44. The method of claim 43, wherein the biological sample is fixed with formaldehyde.

45. The method of claim 43, wherein the biological sample is fixed with methanol.

46. A method of improving predictive value of a biomarker, the method comprising:
(a) obtaining a plurality of images of a high-power field (HPF) generated from a biological sample;
(b) detecting a biomarker in each of the plurality of images;
(c) selecting a sub-plurality of images from the plurality of images of step (a); and
(d) analyzing the sub-plurality of images, thereby improving predictive value of the biomarker.

47. The method of claim 46, wherein the method further comprises generating an area under the ROC (receiver operating characteristics) curve value that is greater than an area under the ROC (receiver operating characteristics) curve value generated when analyzing all of the images.

48. The method of claim 46 or 47, wherein the biomarker comprises PD-1, PD-L1, CD8, FoxP3, CD163, a tumor cell marker, or any combination thereof.

49. The method of claim 48, wherein the tumor cell marker comprises Sox10, S100, or both.

50. The method of any one of claims 46-49, wherein the sub-plurality of images is 30% of the plurality of images of step (a).

51. The method of any one of claims 46-50, wherein the obtaining step (a) comprises performing immunofluorescence microscopy on the biological sample.

52. The method of any one of claims 46-51, wherein the analyzing step (d) comprises:
(i) image acquisition and processing;
(ii) cell segmentation and phenotyping; and
(iii) image normalization.

53. The method of claim 52, wherein the step of image acquisition comprises compiling the plurality of images of a high-power field (HPF) to acquire an image of the whole biological sample.

54. The method of claim 53, wherein the compiling comprises aligning the plurality of images with an overlap.

55. The method of claim 52, wherein the step of cell segmentation and phenotyping comprises identifying a cell type in the biological sample.

56. The method of claim 55, wherein the step of phenotyping comprises detecting expression of the biomarker in the cell type.

57. The method of claim 56, wherein the expression of the biomarker is designated as low, medium, or high.

58. The method of claim 55, wherein the cell type comprises a CD163+macrophage, a CD8+ T cell, a Treg cell (CD8negFoxP3+), a tumor cell, a CD8+FoxP3+ cell, or any combinations thereof.

59. The method of claim 55, wherein the cell type of CD8+FoxP3+PD-1 low/mid is identified as an indicator that the subject will respond to the immunotherapy.

60. The method of claim 55, wherein the cell type of CD163+PD-L1 neg is identified as an indicator that the subject will not respond to the immunotherapy to the same extent as a reference subject that is identified as not having a cell type of CD163+PD-L1 neg.

61. The method of claim 55, wherein the step of cell segmentation and phenotyping further comprises determining a density of the cell type in the biological sample.

62. The method of claim 61, wherein the density of the cell type in the biological sample is determined by analyzing a distance between a cell and another cell.

63. The method of claim 61, wherein the density of the cell type in the biological sample is determined by analyzing a distance between a cell and a tumor-stromal boundary.

64. The method of claim 61, wherein a high density of CD8+FoxP3+ cells is identified as an indicator that the subject will respond to the immunotherapy.

65. The method of claim 52, wherein the step of image normalization comprises calibrating a fluorescence intensity of the biomarker in the plurality of images against a tissue micro array.
